(19) United States
(12) Patent Application Publication
(10) Pub. No.: US 2022/0280690 A1
(43) Pub. Date: Sep. 8, 2022

(54) REINFORCED COLLAGEN DEVICE FOR SOFT TISSUE REPAIR

(71) Applicant: Harbor MedTech, Inc., Lake Forest, CA (US)

(72) Inventors: Keith Elmer Myers, Lake Forest, CA (US); William Jerry Mezger, Coto de Caza, CA (US)

(21) Appl. No.: 17/684,196

(22) Filed: Mar. 1, 2022

Related U.S. Application Data
(60) Provisional application No. 63/155,630, filed on Mar. 2, 2021.

(51) Int. Cl. A61L 27/24; A61L 27/36; A61F 2/00; A61F 2/44; A61F 2/32; A61F 2/30

(52) U.S. Cl. CPC A61L 27/24; A61L 27/3687; A61F 2/0063; A61F 2/44; A61F 2/32; A61F 2/3094; A61F 2002/30461; A61L 2430/10; A61L 2430/02; A61L 2430/38

(57) ABSTRACT

Disclosed herein are embodiments of orthopedic devices. In several embodiments, orthopedic device comprises a biocompatible covering. In several embodiments the biocompatible covering is a collagen-based material. In several embodiments, the collagen-based material is crosslinked using an epoxide-based crosslinking agent (e.g., a diepoxide, triepoxide, etc.). In several embodiments, after a precursor crosslinked collagen-based material is prepared (e.g., by subjecting it to crosslinking conditions), residual crosslinking agent in the precursor material is quenched. In several embodiments, it has been surprisingly found that, by subjecting the precursor crosslinked collagen based material to a quenching reaction (to provide the crosslinked collagen-based material), improved properties are obtained (e.g., lower toxicity lower cytotoxicity, etc.). In several embodiments, the crosslinked collagen-based material is fabricated into an orthopedic device or used to prepare an orthopedic device (e.g., implant).

This application claims the priority benefit under 35 U.S.C. § 119(e) to U.S. Provisional Patent Application Ser. No. 63/155,630, filed Mar. 2, 2021, titled “REINFORCED COLLAGEN DEVICE FOR SOFT TISSUE REPAIR”, the entirety of which is hereby expressly incorporated by reference as if fully set forth herein.

### FIELD OF THE DISCLOSURE

The present disclosure relates to devices, materials comprising such devices, methods of manufacturing such devices and materials, and methods for repair of tendons of the joints and other connective tissue and other tissue defects, and particularly for repair of distal extremity joints.

### BACKGROUND

### Description of the Related Art

Many medical products are composed from human or animal tissue-based materials. Examples of these medical products include, for example, heart valves, vascular grafts, urinary bladder prostheses, tendon prostheses, surgical mesh, and skin substitutes. These products may be composed of animal tissue materials mainly consisting of or having collagen. However, conventional medical products composed from human or animal tissue-based materials can have many problems and drawbacks, which can include excessive time to remodel or heal the injury, rapid degradation of the product or portions thereof, which can have negative consequences on healing, demonstrated inflammatory response to the device (such as, for example, so called “Rice Bodies” products). Additionally, some conventional medical products composed from human or animal tissue-based materials are too thick for use in sensitive tendon locations, particularly those in distal extremity joints. Therefore, there remains a need for improved medical products, for example, for use in soft tissue repair of distal extremity joints.

### SUMMARY OF SOME EXEMPLIFYING EMBODIMENTS

Certain embodiments disclosed herein are directed to orthopedic implants for use in soft tissue repair comprising collagen, and methods of making and using such implants. Some orthopedic implants as described herein are adapted for use in soft tissue repair of a distal extremity joint. Some orthopedic implants as described herein comprise a support element that is covered by a crosslinked collagen-based covering, for example a crosslinked collagen-based covering wrapped at least partially around a braided support element.

Some methods of making an orthopedic implant, such as a distal extremity implant as described herein, comprise providing a collagen-based material, exposing the collagen-based material to crosslinking conditions to provide a crosslinked collagen-based material, and exposing the crosslinked collagen-based material to a quenching agent to provide a treated crosslinked collagen-based material. In some embodiments, the treated crosslinked collagen-based material is coupled with at least a portion of a support element, for example a braided support element. Other methods are directed to making an orthopedic device comprising a treated crosslinked collagen-based material, comprising providing a braided orthopedic implant base structure, and attaching the treated crosslinked collagen-based material to the orthopedic implant base structure. Other methods of making an orthopedic implant, such as a distal extremity implant, comprise providing a collagen-based material, and controllably crosslinking the collagen so that only a portion of the collagen is crosslinked.

In any of the embodiments of a method of making an orthopedic device disclosed herein, the method can include providing a collagen-based material and controllably crosslinking the collagen so that only a portion of the collagen is crosslinked. Any of the embodiments of the implant devices disclosed herein can formed using this method.

Disclosed herein are embodiments of an orthopedic implant for treatment of a distal extremity joint that can include a support element including a first end portion, a second portion, and a middle portion between the first end portion and the second end portion, the middle portion having a first main surface having a first width, a second main surface opposite to the first main surface, a first side edge surface, and a second side edge surface, and a crosslinked collagen-based covering positioned around at least part of a length of the middle portion of the support element so that the crosslinked collagen-based covering covers at least part of the length of the first main surface, the second main surface, and the first side edge of the middle portion of the support element. In some embodiments, the crosslinked collagen-based covering can include a collagenous substrate including collagen strands, a crosslink, and a quenched crosslinking agent. The crosslink can optionally include a crosslinking unit, a first amine, and a second amine, the first amine being part of a first collagen strand of the collagenous substrate and the second amine being part of a second collagen strand of the collagenous substrate, the crosslink being represented by Formula (I):

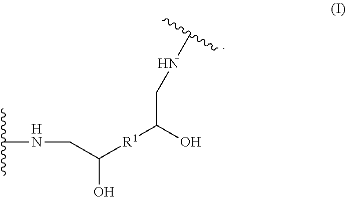

The quenched crosslinking agent can optionally be bonded to the collagenous material through a third amine of the collagenous substrate and can be represented by Formula (II):

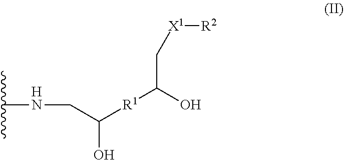

where R¹ can be selected from the group consisting of optionally substituted alkylene, optionally substituted polyether, and optionally substituted polyamino, R² can be selected from the group consisting of optionally substituted alkylene, optionally substituted polyether, and optionally substituted polyamino. X¹ can be selected from the group consisting of —O— and —NH— where each instance of “ [symbol] ” of Formulae (I) and (II) represents a portion of the collagenous substrate. In any embodiments disclosed herein, the support element can include at least one of a thread, a suture, a sheet, a strip, a fabric, and a weave, and/or the support element can include a braided material; wherein the support element can include a mesh material; and/or wherein the support element can include a polymeric surgical mesh material.

Also disclosed herein are embodiments of an orthopedic implant for treatment of a soft tissue defect. In any embodiments, the orthopedic implant can include a braided support element that can include a first end portion, a second portion, and a middle portion between the first end portion and the second end portion, the middle portion having a first main surface having a first width, a second main surface opposite to the first main surface, a first side edge surface, and a second side edge surface, and a crosslinked collagen-based covering positioned around at least part of a length of the middle portion of the support element so that the crosslinked collagen-based covering covers at least part of the length of the first main surface, the second main surface, and the first side edge of the middle portion of the support element, the crosslinked collagen-based covering. In some embodiments, the covering can include a collagenous substrate including collagen strands, a crosslink, and a quenched crosslinking agent. In some embodiments, the crosslink can include a crosslinking unit, a first amine, and a second amine, the first amine being part of a first collagen strand of the collagenous substrate and the second amine being part of a second collagen strand of the collagenous substrate, the crosslink being represented by Formula (I):

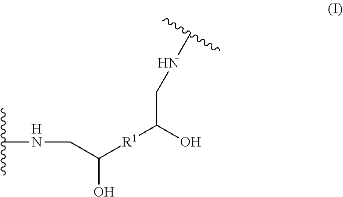

wherein the quenched crosslinking agent can be bonded to the collagenous material through a third amine of the collagenous substrate and can be represented by Formula (II):

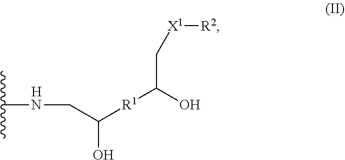

where R¹ can be selected from the group consisting of optionally substituted alkylene, optionally substituted polyether, and optionally substituted polyamino, R² can be selected from the group consisting of optionally substituted alkylene, optionally substituted polyether, and optionally substituted polyamino, and X¹ can be selected from the group consisting of —O— and —NH—, where each instance of “ [symbol] ” of Formulae (I) and (II) represents a portion of the collagenous substrate. In some embodiments, the crosslink can be further represented by Formula (Ia):

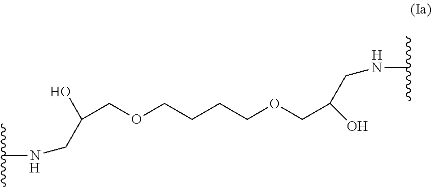

Any embodiments of the methods, implants, and/or devices disclosed herein can include, in additional embodiments, one or more of the following steps, features, components, and/or details, in any combination with any of the other steps, features, components, and/or details of any other embodiments disclosed herein: wherein the orthopedic implant can be sized and configured for use in an abdominal hernia treatment procedure; wherein the orthopedic implant can be configured for tendon, ligament, and/or other tissue repair, support, reconstruction and/or other treatment of a patient's hip; wherein the orthopedic implant can be configured for tendon, ligament, and/or other tissue repair, support, reconstruction and/or other treatment of any of a patient's spine; wherein the crosslinked collagen-based covering does not cover the first end portion or the second end portion of the support element; wherein the crosslinked collagen-based covering covers at least the first main surface, the second main surface, and the first side edge; wherein the crosslinked collagen-based covering can be wrapped around at least the middle portion of the support element; wherein the crosslinked collagen-based covering can include a sheet of the collagenous substrate that can be wrapped around at least the first main surface, the second main surface, and the first side edge surface of the middle portion of the support element; wherein the crosslinked collagen-based covering can include a sheet of the collagenous substrate that can be wrapped continuously around the first main surface, the second main surface, the first side edge surface, and the second side edge surface of the middle portion of the support element; wherein the crosslinked collagen-based covering can include a sheet of the collagenous substrate that has at least one fold along a length thereof, the fold being configured to facilitate a more uniform height or thickness of the orthopedic implant along the middle portion of the support element; wherein the crosslinked collagen-based covering can include at least one cut along the at least one fold; wherein the at least one fold can be aligned with the first side edge surface; wherein the crosslinked collagen-based covering can include at least one channel having a reduced thickness along the at least one fold, the reduced thickness being less than a thickness of the crosslinked collagen-based covering adjacent to the at least one channel; wherein the reduced thickness of the channel can be 0.1 mm, or approximately 0.1 mm, or from 0.05 mm to 1.5 mm; and/or wherein the thickness of the crosslinked collagen-based covering adjacent to the at least one channel can be 0.5 mm or approximately 0.5 mm, or from 0.3 mm to 0.7 mm.

Any embodiments of the methods, implants, and/or devices disclosed herein can include, in additional embodiments, one or more of the following steps, features, components, and/or details, in any combination with any of the other steps, features, components, and/or details of any other embodiments disclosed herein: wherein the reduced thickness of the channel can be from 10% or approximately 10% to 50% or approximately 50% of the thickness of the crosslinked collagen-based covering adjacent to the at least one channel; wherein the reduced thickness of the channel can be from 15% or approximately 15% to 30% or approximately 30% of the thickness of the crosslinked collagen-based covering adjacent to the at least one channel; wherein the reduced thickness of the channel can be 20%, or approximately 20%, or from 10% or approximately 10% to 30% or approximately 30% of the thickness of the crosslinked collagen-based covering adjacent to the at least one channel; wherein the crosslinked collagen-based covering can be attached to the support element using one or more sutures; wherein the support element can include can be wider in the middle portion than in the first or second end portions; wherein the first width of the middle portion of the support element can be greater than a width of the first end portion and/or the second end portion of the support element; wherein the support element has a length from an end of the first end portion to an end of the second end portion, and the crosslinked collagen-based covering has a length parallel to the first side edge surface and the second side edge surface; wherein the length of the support element can be greater than the length of the crosslinked collagen-based covering; wherein the implant can include multiple lines of sutures extending along a length of the crosslinked collagen-based covering; wherein the crosslinked collagen-based covering can be configured to be cut in an anatomical shape to match a shape of a natural tendinous structure requiring surgical repair when attached to the support element; wherein the crosslinked collagen-based covering covers an entire periphery of the middle portion of the support element along a length of the support element that can be covered by the crosslinked collagen-based covering; wherein the implant can be configured to cover a joint or a sliding tendinous structure; wherein the orthopedic implant can be sized and configured for use in an ankle joint; wherein the R¹ can be represented by a structure selected from the group consisting of: —(CH₂)ₐ—(O—(CH₂)_{b})_{c}—O—(CH₂)_{d}—, —(CH₂)ₐ—(NH—(CH₂)_{b})_{c}—NH—(CH₂)_{d}—, and —(CH₂)ₐ—, where each of a, b, c, and d can be independently an integer equal to 0, 1, 2, 3, 4, 5, 6, 7, or 8; wherein the orthopedic implant can be configured for tendon, ligament, and/or other tissue repair, support, reconstruction and/or other treatment in any distal extremity joint in the body; wherein the orthopedic implant can be configured for tendon, ligament, and/or other tissue repair, support, reconstruction and/or other treatment of a patient's ankle, knee, wrist, hand, or foot; wherein R¹ can be represented by —CH₂—O—(CH₂)_{b}—O—CH₂— and b can be 4.

In any embodiments disclosed herein, the R² can be represented by a structure selected from the group consisting of: —(CH₂)ₐ—(O—(CH₂)_{b})_{c}—O—(CH₂)_{d}—H, —(CH₂)ₐ—(NH—(CH₂)_{b})_{c}—NH—(CH₂)_{d}—H, and —(CH₂)ₐ—H, where each of a, b, c, and d can be independently an integer equal to 0, 1, 2, 3, 4, 5, 6, 7, or 8. In any embodiments disclosed herein, the quenched crosslinking agent can be represented by Formula (IIa):

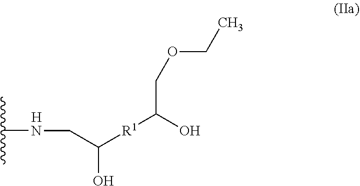

Also disclosed herein are embodiments of a method of making an orthopedic implant that can include providing a collagen-based material, exposing the collagen-based material to crosslinking conditions to provide a crosslinked collagen-based material, exposing the crosslinked collagen-based material to a quenching agent to provide a treated crosslinked collagen-based material, and coupling the treated crosslinked collagen-based material with at least a portion of a braided support element. In some embodiments, coupling the treated crosslinked collagen-based material with at least a portion of the braided support element can include wrapping a sheet of the treated crosslinked collagen-based material around at least part of a length of a middle portion of the support element so that the treated crosslinked collagen-based material covers at least a first main surface, a second main surface, and a side edge surface of at least part of a length of the middle portion of the support element. In some embodiments, the middle portion can be between a first end portion and a second end portion of the support element.

Any embodiments of the methods, implants, and/or devices disclosed herein can include, in additional embodiments, one or more of the following steps, features, components, and/or details, in any combination with any of the other steps, features, components, and/or details of any other embodiments disclosed herein: forming at least one fold in the sheet of the treated crosslinked collagen-based material along a length thereof, and wrapping the sheet of the treated crosslinked collagen-based material around at least the middle portion of the support element; forming at least one fold in the sheet of the treated crosslinked collagen-based material along a length thereof, and wrapping the sheet of the treated crosslinked collagen-based material around at least the middle portion of the support element so that the at least one fold can be positioned adjacent to a side edge of the middle portion of the support element; wherein forming at least one fold in the sheet of the treated crosslinked collagen-based material along a length thereof can include forming a cut line through less than an entire thickness of the sheet of the treated crosslinked collagen-based material along the length of the material and folding the sheet of the treated crosslinked collagen-based material along the cut line; wherein a thickness of the sheet of the treated crosslinked collagen-based material along the cut line can be less than half of a thickness of the sheet of the treated crosslinked collagen-based material adjacent to the cut line; wherein the thickness of the sheet of the treated crosslinked collagen-based material along the cut line can be 20%, or approximately 20%, or from 10% to 30% of the thickness of the sheet of the treated crosslinked collagen-based material adjacent to the cut line; wherein the thickness of the sheet of the treated crosslinked collagen-based material along the cut line can be 0.1 mm or approximately 0.1 mm, or from 0.05 mm to 0.15 mm, and the thickness of the sheet of the treated crosslinked collagen-based material adjacent to the cut line can be 0.5 mm or approximately 0.5 mm, or from 0.3 mm to 0.7 mm; wherein forming the cut line through less than the entire thickness of the sheet of the treated crosslinked collagen-based material along the length of the material can include cutting the sheet along the cut line with a knife or a laser; wherein forming the cut line through less than the entire thickness of the sheet of the treated crosslinked collagen-based material along the length of the treated crosslinked collagen-based material can include removing a portion of the treated crosslinked collagen-based material along the cut line to form a channel along the length of the treated crosslinked collagen-based material; wherein exposing the collagen-based material to crosslinking conditions includes immersing the collagen-based material in a buffered solution having a pH that can be between 8.9 or about 8.9 to 9.5 or about 9.5; wherein exposing the collagen-based material to crosslinking conditions includes immersing the collagen-based material in a buffered solution having a pH that can be between 4.2 or about 4.2 to 4.8 or about 4.8; wherein exposing the collagen-based material to crosslinking conditions includes exposing the collagen-based material to a crosslinking agent at a concentration between 1% or about 1% w/v to 10% or about 10% w/v; wherein exposing the collagen-based material to crosslinking conditions includes exposing the collagen-based material to a crosslinking agent at a concentration of 4% or about 4% w/v, or from 3% to 5% w/v; wherein exposing the collagen-based material to crosslinking conditions includes exposing the collagen-based material to one or more crosslinking agents independently selected from the group consisting of glycol diglycidyl ether, glycerol diglycidyl ether, glycerol triglycidyl ether, and butanediol diglycidyl ether; wherein exposing the collagen-based material to crosslinking conditions includes exposing the collagen-based material to 1,4 butanediol diglycidyl ether; wherein the treated crosslinked collagen-based material has a first percentage of free amine groups on the collagen strands and the tailorably crosslinked collagen-based material has a second percentage of free amine groups on the collagen strands; wherein the second percentage can be lower than the first percentage; wherein the amount of free amines in the tailorably crosslinked collagen-based material can be between 50% or about 50% to 85% or about 85%; and/or wherein the amount of free amines in the tailorably crosslinked collagen-based material can be between 60% or about 60% to 75% or about 75%.

Also disclosed herein are embodiments of a method of making an orthopedic implant including a treated crosslinked collagen-based material. In some embodiments, the method can include providing a braided orthopedic implant base structure and attaching the treated crosslinked collagen-based material to the braided orthopedic implant base structure, wherein the treated crosslinked collagen-based material can be prepared by exposing a collagen material to a crosslinking solution including a crosslinking agent to provide a crosslinked collagen-based material and exposing the collagen-based material to a quenching agent to provide the treated crosslinked collagen-based material.

Any embodiments of the methods, implants, and/or devices disclosed herein can include, in additional embodiments, one or more of the following steps, features, components, and/or details, in any combination with any of the other steps, features, components, and/or details of any other embodiments disclosed herein: wherein the crosslinking solution can be a buffered solution with a pH between 8.0 or about 8.0 to 10.5 or about 10.5; wherein the crosslinking agent can be a diepoxide; wherein the concentration of each of the crosslinking agent can be between 1% or about 1% w/v to 10% or about 10% w/v; wherein the concentration of each of the first crosslinking agent and the second crosslinking agent can be 4% or about 4% w/v; wherein the collagen material can be exposed to the crosslinking solution for 150 hours or about 150 hours to 159 hours or about 159 hours; wherein the crosslinking agent can be selected from the group consisting of glycol diglycidyl ether, glycerol diglycidyl ether, glycerol triglycidyl ether, and butanediol diglycidyl ether; and/or wherein the first and second crosslinking agents are 1,4 butanediol diglycidyl ether.

Also disclosed herein are embodiments of an orthopedic implant made by any of the embodiments the methods disclosed herein. Also disclosed herein are embodiments of a method of treating a tissue defect substantially as hereinbefore described and/or shown in the accompanying drawings. Also disclosed herein are embodiments of a method of implanting a medical device substantially as hereinbefore described or shown in the accompanying drawings. Also disclosed herein are embodiments of a medical implant substantially as hereinbefore described and/or shown in the accompanying drawings.

The above embodiments, and other embodiments directed to other orthopedic and non-orthopedic implants, medical products, and methods of manufacture or use, are described throughout this specification and the appended claims.

### BRIEF DESCRIPTION OF THE DRAWINGS

FIGS. 1A-1D depict embodiments of orthopedic implants as disclosed elsewhere herein.

FIGS. 2A-2C depict embodiments of crosslinked collagen-based coverings as disclosed elsewhere herein.

FIG. 3 is a flow chart depicting an embodiment of a method of synthesizing a crosslinked collagen-based material.

FIG. 4 is a flow chart depicting another embodiment of a method of synthesizing a crosslinked collagen-based material.

FIG. 5 is a flow chart detailing optional steps that may be used in preparing a crosslinked collagen-based material as described in FIG. 3.

FIG. 6 is a flow chart detailing optional steps that may be used in preparing a crosslinked collagen-based material as described in FIG. 4.

### DETAILED DESCRIPTION OF SOME EXEMPLIFYING EMBODIMENTS

Disclosed herein are embodiments of orthopedic devices comprising collagen-based materials. The term “orthopedic,” as it is used herein, is meant to refer to devices that are configured to treat or are related to the treatment of deformities of bones, muscles, ligaments, tendons, and/or other connective tissue. The term “soft tissue,” as used herein, is meant to refer to tissues that connect, support, or surround other structures and organs of the body. Nonlimiting examples of soft tissue include muscles, tendons, ligaments, fascia, nerves, fibrous tissues, fat, blood vessels, and synovial membranes, any or all of which are meant to be included in any use of the term soft tissue herein.

Some embodiments disclosed herein are directed to a method of making an orthopedic implant including shaping a treated crosslinked collagen-based material to provide at least a portion of an orthopedic implant, wherein the treated crosslinked collagen-based material is made by exposing a collagen material to a crosslinking solution comprising a crosslinking agent to provide a crosslinked collagen-based material, and exposing the collagen-based material to a quenching agent to provide the treated crosslinked collagen-based material. Some embodiments disclosed herein are directed to a method of making an orthopedic implant comprising a treated crosslinked collagen-based material, including providing an orthopedic implant base structure and attaching the treated crosslinked collagen-based material to the orthopedic implant base structure. In some embodiments, the treated crosslinked collagen-based material can be prepared by crosslinking a collagen material with a crosslinking agent to provide a precursor crosslinked collagen-based material and by quenching any unreacted reactive groups of the crosslinking agent on or within the collagen material to provide the treated crosslinked collagen-based material.

In several embodiments, a crosslinked collagen-based material is formed (e.g., shaped, cut, etc.) into a covering. In several embodiments, the covering is used fixed, adhered, or placed over an orthopedic implant (e.g., a suture or another orthopedic device), or the covering may be used without fixing, adhering or placing it over another orthopedic implant. In several embodiments, using an orthopedic device comprising a crosslinked collagen-based covering, the device has improved biocompatibility upon implantation. In several embodiments, using an orthopedic device comprising a crosslinked collagen-based covering as disclosed herein helps reduce and/or avoid undesired responses to the orthopedic device once implanted (e.g., cytotoxicity, toxicity, foreign body granuloma, scar tissue formation, capsule formation, etc.). In several embodiments, the crosslinked collagen-based material comprises a collagen substrate that has been crosslinked using an epoxide-based crosslinking agent (e.g., a diepoxide, triepoxide, etc.). In several embodiments, after the crosslinked collagen-based material is prepared (e.g., by subjecting it to crosslinking conditions), the crosslinking agent is quenched. It has been surprisingly found that, by subjecting a crosslinked collagen-based material to a quenching reaction after preparation, improved properties are obtained (e.g., lower toxicity lower cytotoxicity, etc.). The following description provides context and examples, but should not be interpreted to limit the scope of the inventions covered by the claims that follow in this specification or in any other application that claims priority to this specification. No single component or collection of components or steps is essential or indispensable. Any feature, structure, component, material, step, or method that is described and/or illustrated in any embodiment in this specification can be used with or instead of any feature, structure, component, material, step, or method that is described and/or illustrated in any other embodiment in this specification.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “comprising” as used herein is synonymous with “including,” “containing,” or “characterized by,” and is inclusive or open-ended and does not exclude additional, unrecited elements or method steps; the term “having” should be interpreted as “having at least;” the term “includes” should be interpreted as “includes but is not limited to;” the term “example” is used to provide exemplary instances of the item in discussion, not an exhaustive or limiting list thereof; and use of terms like “preferably,” “preferred,” “desired,” or “desirable,” and words of similar meaning should not be understood as implying that certain features are critical, essential, or even important to the structure or function of the invention, but instead as merely intended to highlight alternative or additional features that may or may not be utilized in a particular embodiment of the invention. In addition, the term “comprising” is to be interpreted synonymously with the phrases “having at least” or “including at least”. When used in the context of a process, the term “comprising” means that the process includes at least the recited steps, but may include additional steps. When used in the context of a compound, composition or device, the term “comprising” means that the compound, composition or device includes at least the recited features or components, but may also include additional features or components. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Additionally, the phrase “consisting essentially of” will be understood to include those elements specifically recited and those additional elements that do not materially affect the basic and novel characteristics of the claimed technology. The phrase “consisting of” excludes any element not specified.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

“Treat” or “treating” or “treatment” refers to any type of action that imparts a modulating effect or therapeutic effect, which, for example, can be a beneficial effect, to a subject afflicted with an injury, disorder, disease or illness, including improvement in the condition of the subject, delay or reduction in the progression of the condition, and/or change in clinical parameters, injury or illness, curing the injury, etc. Treatment may include reduction of the symptoms of an injury or structural damage to the body (e.g., pain, reduced range of motion, stiffness, etc., resulting from an injury or damage to a joint)

The “patient” or “subject” treated as disclosed herein is, in some embodiments, a human patient, although it is to be understood that the principles of the presently disclosed subject matter indicate that the presently disclosed subject matter is effective with respect to all vertebrate species, including mammals, which are intended to be included in the terms “subject” and “patient.” Suitable subjects are generally mammalian subjects. The subject matter described herein finds use in research as well as veterinary and medical applications. The term “mammal” as used herein includes, but is not limited to, humans, non-human primates, cattle, sheep, goats, pigs, horses, cats, dog, rabbits, rodents (e.g., rats or mice), monkeys, etc. Human subjects include neonates, infants, juveniles, adults and geriatric subjects.

Whenever a group is described as being “optionally substituted” that group may be unsubstituted or substituted with one or more of the indicated substituents. Likewise, when a group is described as being “unsubstituted or substituted” (or “substituted or unsubstituted”) if substituted, the substituent(s) may be selected from one or more the indicated substituents. If no substituents are indicated, it is meant that the indicated “optionally substituted” or “substituted” group may be substituted with one or more group(s) individually and independently selected from alkyl, alkenyl, alkynyl, cycloalkyl, cycloalkenyl, cycloalkyl(alkyl), hydroxy, alkoxy, acyl, cyano, halogen, C-carboxy, O-carboxy, nitro, sulfenyl, haloalkyl, haloalkoxy, an amino, a mono-substituted amine group, a di-substituted amine group, a mono-substituted amine(alkyl), a di-substituted amine(alkyl), a diamino-group, a polyamino, a diether-group, and a polyether-.

As used herein, “Cₐ to C_{b}” in which “a” and “b” are integers refer to the number of carbon atoms in a group. The indicated group can contain from “a” to “b”, inclusive, carbon atoms. Thus, for example, a “C₁ to C₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—. If no “a” and “b” are designated, the broadest range described in these definitions is to be assumed.

If two “R” groups are described as being “taken together” the R groups and the atoms they are attached to can form a cycloalkyl, cycloalkenyl, aryl, heteroaryl or heterocycle. For example, without limitation, if R^{a} and R^{b} of an NR^{a}R^{b} group are indicated to be “taken together,” it means that they are covalently bonded to one another to form a ring:

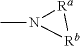

As used herein, the term “alkyl” refers to a fully saturated aliphatic hydrocarbon group. The alkyl moiety may be branched or straight chain. Examples of branched alkyl groups include, but are not limited to, iso-propyl, sec-butyl, t-butyl and the like. Examples of straight chain alkyl groups include, but are not limited to, methyl, ethyl, n-propyl, n-butyl, n-pentyl, n-hexyl, n-heptyl and the like. The alkyl group may have 1 to 30 carbon atoms (whenever it appears herein, a numerical range such as “1 to 30” refers to each integer in the given range; e.g., “1 to 30 carbon atoms” means that the alkyl group may consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 carbon atoms, although the present definition also covers the occurrence of the term “alkyl” where no numerical range is designated). The “alkyl” group may also be a medium size alkyl having 1 to 12 carbon atoms. The “alkyl” group could also be a lower alkyl having 1 to 6 carbon atoms. An alkyl group may be substituted or unsubstituted. By way of example only, “C₁-C₅ alkyl” indicates that there are one to five carbon atoms in the alkyl chain, i.e., the alkyl chain is selected from methyl, ethyl, n-propyl, isopropyl, n-butyl, isobutyl, tert-butyl, pentyl (branched and straight-chained), etc. Typical alkyl groups include, but are in no way limited to, methyl, ethyl, propyl, isopropyl, butyl, isobutyl, tertiary butyl, pentyl and hexyl.

As used herein, the term “alkylene” refers to a bivalent fully saturated straight chain aliphatic hydrocarbon group. Examples of alkylene groups include, but are not limited to, methylene, ethylene, propylene, butylene, pentylene, hexylene, heptylene and octylene. An alkylene group may be represented by [symbol] , followed by the number of carbon atoms, followed by a “*”. For example,

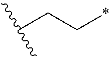 [SMILES: *CC*]

to represent ethylene. The alkylene group may have 1 to 30 carbon atoms (whenever it appears herein, a numerical range such as “1 to 30” refers to each integer in the given range; e.g., “1 to 30 carbon atoms” means that the alkyl group may consist of 1 carbon atom, 2 carbon atoms, 3 carbon atoms, etc., up to and including 30 carbon atoms, although the present definition also covers the occurrence of the term “alkylene” where no numerical range is designated). The alkylene group may also be a medium size alkyl having 1 to 12 carbon atoms. The alkylene group could also be a lower alkyl having 1 to 6 carbon atoms. An alkylene group may be substituted or unsubstituted. For example, a lower alkylene group can be substituted by replacing one or more hydrogen of the lower alkylene group and/or by substituting both hydrogens on the same carbon with a C₃₋₆ monocyclic cycloalkyl group

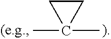

The term “alkenyl” used herein refers to a monovalent straight or branched chain radical of from two to twenty carbon atoms containing a carbon double bond(s) including, but not limited to, 1-propenyl, 2-propenyl, 2-methyl-1-propenyl, 1-butenyl, 2-butenyl and the like. An alkenyl group may be unsubstituted or substituted.

The term “alkynyl” used herein refers to a monovalent straight or branched chain radical of from two to twenty carbon atoms containing a carbon triple bond(s) including, but not limited to, 1-propynyl, 1-butynyl, 2-butynyl and the like. An alkynyl group may be unsubstituted or substituted.

As used herein, “cycloalkyl” refers to a completely saturated (no double or triple bonds) mono- or multi-cyclic (such as bicyclic) hydrocarbon ring system. When composed of two or more rings, the rings may be joined together in a fused, bridged or spiro fashion. As used herein, the term “fused” refers to two rings which have two atoms and one bond in common. As used herein, the term “bridged cycloalkyl” refers to compounds wherein the cycloalkyl contains a linkage of one or more atoms connecting non-adjacent atoms. As used herein, the term “spiro” refers to two rings which have one atom in common and the two rings are not linked by a bridge. Cycloalkyl groups can contain 3 to 30 atoms in the ring(s), 3 to 20 atoms in the ring(s), 3 to 10 atoms in the ring(s), 3 to 8 atoms in the ring(s) or 3 to 6 atoms in the ring(s). A cycloalkyl group may be unsubstituted or substituted. Examples of mono-cycloalkyl groups include, but are in no way limited to, cyclopropyl, cyclobutyl, cyclopentyl, cyclohexyl, cycloheptyl and cyclooctyl. Examples of fused cycloalkyl groups are decahydronaphthalenyl, dodecahydro-1H-phenalenyl and tetradecahydroanthracenyl; examples of bridged cycloalkyl groups are bicyclo[1.1.1]pentyl, adamantanyl and norbornanyl; and examples of spiro cycloalkyl groups include spiro [3.3]heptane and spiro [4.5]decane.

As used herein, “cycloalkenyl” refers to a mono- or multi-cyclic (such as bicyclic) hydrocarbon ring system that contains one or more double bonds in at least one ring; although, if there is more than one, the double bonds cannot form a fully delocalized pi-electron system throughout all the rings (otherwise the group would be “aryl,” as defined herein). Cycloalkenyl groups can contain 3 to 10 atoms in the ring(s), 3 to 8 atoms in the ring(s) or 3 to 6 atoms in the ring(s). When composed of two or more rings, the rings may be connected together in a fused, bridged or spiro fashion. A cycloalkenyl group may be unsubstituted or substituted.

As used herein, “cycloalkyl(alkyl)” refer to a cycloalkyl group connected, as a substituent, via a lower alkylene group. The lower alkylene and cycloalkyl group of a cycloalkyl(alkyl) may be substituted or unsubstituted.

As used herein, the term “hydroxy” refers to a —OH group.

As used herein, “alkoxy” refers to the Formula —OR wherein R is an alkyl, an alkenyl, an alkynyl, a cycloalkyl, a cycloalkenyl, aryl, heteroaryl, heterocyclyl, cycloalkyl(alkyl), aryl(alkyl), heteroaryl(alkyl) or heterocyclyl(alkyl) is defined herein. A non-limiting list of alkoxys are methoxy, ethoxy, n-propoxy, 1-methylethoxy (isopropoxy), n-butoxy, iso-butoxy, sec-butoxy, tert-butoxy, phenoxy and benzoxy. An alkoxy may be substituted or unsubstituted.

As used herein, “acyl” refers to a hydrogen, alkyl, alkenyl, alkynyl, aryl, heteroaryl, heterocyclyl, aryl(alkyl), heteroaryl(alkyl) and heterocyclyl(alkyl) connected, as substituents, via a carbonyl group. Examples include formyl, acetyl, propanoyl, benzoyl and acryl. An acyl may be substituted or unsubstituted.

As used herein, a “cyano” group refers to a “—CN” group.

The term “halogen atom” or “halogen” as used herein, means any one of the radio-stable atoms of column 7 of the Periodic Table of the Elements, such as, fluorine, chlorine, bromine and iodine.

An “O-carboxy” group refers to a “RC(═O)O—” group in which R can be hydrogen, an alkyl, an alkenyl, an alkynyl, a cycloalkyl, a cycloalkenyl, or cycloalkyl(alkyl), as defined herein. An O-carboxy may be substituted or unsubstituted.

The terms “ester” and “C-carboxy” refer to a “—C(═O)OR” group in which R can be the same as defined with respect to O-carboxy. An ester and C-carboxy may be substituted or unsubstituted.

A “nitro” group refers to an “—NO₂” group.

A “sulfenyl” group refers to an “—SR” group in which R can be hydrogen, an alkyl, an alkenyl, an alkynyl, a cycloalkyl, a cycloalkenyl, cycloalkyl(alkyl), or aryl(alkyl). A sulfenyl may be substituted or unsubstituted.

As used herein, “haloalkyl” refers to an alkyl group in which one or more of the hydrogen atoms are replaced by a halogen (e.g., mono-haloalkyl, di-haloalkyl, tri-haloalkyl and polyhaloalkyl). Such groups include but are not limited to, chloromethyl, fluoromethyl, difluoromethyl, trifluoromethyl, 1-chloro-2-fluoromethyl, 2-fluoroisobutyl and pentafluoroethyl. A haloalkyl may be substituted or unsubstituted.

As used herein, “haloalkoxy” refers to an alkoxy group in which one or more of the hydrogen atoms are replaced by a halogen (e.g., mono-haloalkoxy, di-haloalkoxy and tri-haloalkoxy). Such groups include but are not limited to, chloromethoxy, fluoromethoxy, difluoromethoxy, trifluoromethoxy, 1-chloro-2-fluoromethoxy and 2-fluoroisobutoxy. A haloalkoxy may be substituted or unsubstituted.

The terms “amino” and “unsubstituted amino” as used herein refer to a —NH₂ group.

A “mono-substituted amine” group refers to a “—NHRA” group in which R_{A} can be an alkyl, an alkenyl, an alkynyl, a cycloalkyl, a cycloalkenyl, or cycloalkyl(alkyl), as defined herein. The R_{A} may be substituted or unsubstituted. A mono-substituted amine group can include, for example, a mono-alkylamine group, a mono-C₁-C₆ alkylamine group, and the like. Examples of mono-substituted amine groups include, but are not limited to, —NH(methyl), —NH(propyl) and the like.

A “di-substituted amine” group refers to a “—NR_{A}R_{B}” group in which R_{A} and R_{B} can be independently an alkyl, an alkenyl, an alkynyl, a cycloalkyl, a cycloalkenyl, or cycloalkyl(alkyl), as defined herein. R_{A} and R_{B} can independently be substituted or unsubstituted. A di-substituted amine group can include, for example, a di-alkylamine group, a di-C₁-C₆ alkylamine group, and the like. Examples of di-substituted amine groups include, but are not limited to, —N(methyl)₂, —N(propyl)(methyl), —N(ethyl)(methyl) and the like.

As used herein, “mono-substituted amine(alkyl)” group refers to a mono-substituted amine as provided herein connected, as a substituent, via a lower alkylene group. A mono-substituted amine(alkyl) may be substituted or unsubstituted. A mono-substituted amine(alkyl) group can include, for example, a mono-alkylamine(alkyl) group, a mono-C₁-C₆ alkylamine(C₁-C₆ alkyl) group, and the like. Examples of mono-substituted amine(alkyl) groups include, but are not limited to, —CH₂NH(methyl), —CH₂NH(ethyl), —CH₂CH₂NH(methyl), —CH₂CH₂NH(ethyl) and the like.

As used herein, “di-substituted amine(alkyl)” group refers to a di-substituted amine as provided herein connected, as a substituent, via a lower alkylene group. A di-substituted amine(alkyl) may be substituted or unsubstituted. A di-substituted amine(alkyl) group can include, for example, a dialkylamine(alkyl) group, a di-C₁-C₆ alkylamine(C₁-C₆ alkyl) group, and the like. Examples of di-substituted amine(alkyl)groups include, but are not limited to, —CH₂N(methyl)₂, —CH₂N(ethyl)(methyl), —CH₂N(ethyl)(ethyl), —CH₂CH₂N(methyl)₂, and the like.

As used herein, the term “diamino-” denotes an a “—N(R_{A})R_{B}—N(R_{C})(R_{D})” group in which R_{A}, R_{C}, and R_{D} can be independently a hydrogen, an alkyl, an alkenyl, an alkynyl, a cycloalkyl, a cycloalkenyl, or cycloalkyl(alkyl), as defined herein, and wherein R_{B} connects the two “N” groups and can be (independently of R_{A}, R_{C}, and R_{D}) a substituted or unsubstituted alkylene group. R_{A}, R_{B}, R^{C}, and R_{D} can independently further be substituted or unsubstituted.

As used herein, the term “polyamino” means a “—R_{E}—(N(R_{A})R_{B}—)ₙ—N(R_{C})(R_{D})” or a “—R_{E}—(N(R_{A})R_{B}—)ₙ—N(R_{C})—R_{F}—” group where the polyamino spans two structures. R_{A}, R_{B}, R^{C}, and R_{D} are as disclosed elsewhere herein and R_{E} and R_{F} are each C₁₋₆ alkylene or a direct bond. For illustration, the term polyamino can comprise —CH₂—N(R_{A})alkyl-N(R_{A})alkyl-N(R_{A})alkyl-N(R_{A})alkyl-N(R_{C})—CH₂CH₂— where R_{E} is CH₂, R_{B} is alkyl, and R_{F} is —CH₂CH₂—. In some embodiments, the alkyl of the polyamino is as disclosed elsewhere herein. While this example has only 4 repeat units, the term “polyamino” may consist of 1, 2, 3, 4, 5, 6, 7, 8, 9, or 10 repeat units (e.g., n may be 1, 2, 3, 4, 5, 6, 7, 8, 9, or 10). R_{A}, R_{C}, and R_{D}, where present, can be independently a hydrogen, an alkyl, an alkenyl, an alkynyl, or a cycloalkyl, as defined herein, and wherein R_{B} connects the two “N” groups and can be (independently of R_{A}, R_{C}, and R_{D}) a substituted or unsubstituted alkylene group. R_{A}, R_{C}, and R_{D} can independently further be substituted or unsubstituted. As noted here, the polyamino comprises amine groups with intervening alkyl groups (where alkyl is as defined elsewhere herein).

As used herein, the term “polyether” denotes a repeating —R_{E}—(OR_{B}—)ₙOR_{D} group or a “—(R_{E})—(OR_{B}—)ₙO—R_{F}—” group where the polyether spans two structures. R_{B}, R_{D}, R_{E}, and R_{F} are as disclosed elsewhere herein. For instance, R_{E} and R_{F} may each be C₁₋₆ alkylene or a direct bond. For illustration, the term polyether can comprise —Oalkyl-Oalkyl-Oalkyl-Oalkyl-OR_{A}. For further illustration, the term polyether can comprise —CH₂—Oalkyl-Oalkyl-O—CH₂CH₂— where R_{E} is CH₂, R_{B} is alkyl, and R_{F} is —CH₂CH₂—. In some embodiments, the alkyl of the polyether is as disclosed elsewhere herein. While this example has only 2 repeat units, the term “polyether” may consist of 1, 2, 3, 4, 5, 6, 7, 8, 9, or 10 repeat units (e.g., n may be 1, 2, 3, 4, 5, 6, 7, 8, 9, or 10). R_{D} can be a hydrogen, an alkyl, an alkenyl, an alkynyl, a cycloalkyl, or a cycloalkenyl, as defined herein. R_{B} can be a substituted or unsubstituted alkylene group. R_{A} can independently further be substituted or unsubstituted. As noted here, the polyether comprises ether groups with intervening alkyl groups (where alkyl is as defined elsewhere herein and can be optionally substituted).

Where the number of substituents is not specified (e.g., haloalkyl), there may be one or more substituents present. For example, “haloalkyl” may include one or more of the same or different halogens.

As used herein, a radical indicates species with a single, unpaired electron such that the species containing the radical can be covalently bonded to another species. Hence, in this context, a radical is not necessarily a free radical. Rather, a radical indicates a specific portion of a larger molecule. The term “radical” can be used interchangeably with the term “group.”

As used herein, the term “collagen strand” refers to tropocollagen, collagen fibrils and/or collagen fibers. Collagenous substrates (e.g., collagen substrates) may comprise a matrix of collagen strands. Collagen strands have pendant amine (—NH₂) and carboxylic acid (—COOH) groups which are reactive (e.g., to crosslinking agents). These amines and carboxylic acid groups are readily crosslinked between collagen strands with various crosslinking agents to form structures with improved medial properties. Crosslinking can be performed by taking advantage of pendant reactive groups on the collagen strand.

As used herein, the term “degradation time” refers to the amount of time it takes for a collagen-based material to completely degrade or to degrade to such an extent that it no longer serves the purpose for which it was medically intended. When degradation times are provided herein, those degradation rates may be performed under conditions described in the Examples, for example, under pronase degradation conditions.

### Introduction

Many medical products are composed from human or animal tissue-based materials. Examples of these medical products include, for example, heart valves, vascular grafts, urinary bladder prostheses, tendon prostheses, hernia patches, surgical mesh, and skin substitutes. An illustration of a specific human or animal tissue-based product is the heart valve prosthesis. Heart valve prostheses are typically made from either porcine aortic valves or bovine pericardium. Such valves are typically made by pretreating the tissue with glutaraldehyde or other crosslinking agents and sewing the tissue into a flexible metallic alloy or polymeric stent. These tissue starting materials (which may be from any mammal, including humans) mainly consist of or comprise collagen, which provides the tissues with their needed mechanical strength and flexibility.

Collagen-based materials, including whole tissue, are finding increased use in the manufacture of biomedical devices, such as prosthetic implants. Collagen is a naturally occurring protein featuring good biocompatibility. It is the major structural component of vertebrates, forming extracellular fibers or networks in practically every tissue of the body, including skin, bone, cartilage, and blood vessels. As a natural component of the extracellular matrix, collagen provides a good physiological, isotropic environment that promotes the growth and function of different cell types and facilitates rapid overgrowth of host tissue in medical devices after implantation.

Three main types of collagen-based materials can be identified, based on the differences in the purity and integrity of the collagen fiber bundle network initially present in the material. The first type includes whole tissue including non-collagenous substances or cells. As a result of using whole tissue, the naturally occurring composition and the native strength and structure of the collagen fiber bundle network are preserved. Whole tissue xenografts have been used in construction of heart valve prostheses and in many other biomedical prostheses. However, the presence of soluble proteins, glycoproteins, glycosaminoglycans, and cellular components in such whole tissue xenografts may induce an immunological response of the host organism to the implant. The second type of collagen-based material includes only the collagen matrix without the non-collagenous substances. The naturally occurring structure of the collagen fiber bundle network is thus preserved, but the antigenicity of the material is reduced. The fibrous collagen materials obtained by removing the antigenic non-collagenous substances will generally have suitable mechanical properties. The third type of collagen-based material is purified fibrous collagen. Purified collagen is obtained from whole tissue by first dispersing or solubilizing the whole tissue by either mechanical or enzymatic action. The collagen dispersion or solution is then reconstituted by either air drying, lyophilizing, or precipitating out the collagen. A variety of geometrical shapes like sheets, tubes, sponges or fibers can be obtained from the collagen in this way. The resulting materials, however, do not have the mechanical strength of the naturally occurring fibrous collagen structure.

A major problem in the use of collagen-based materials for implantation, and especially whole tissue xenografts in which the donor and recipient are phylogenetically distant, is that these materials are prone to acute rejection. This is a rapid and violent immunological reaction that leads to the destruction of the xenograft. In order to use collagen-based materials in manufactured medical devices, particularly bioprosthetic implants, their durability and in vivo performance typically need to be protected from an acute immunological reaction. Crosslinking the collagen-based materials may help suppress the antigenicity of the material in order to prevent the acute rejection reaction. In addition, crosslinking is used to preserve or even improve mechanical properties and to enhance resistance to degradation.

Several chemical crosslinking methods for collagen-based materials are known. These methods involve the reaction of a bifunctional reagent with the amine groups of lysine or hydroxylysine residues on different polypeptide chains or the activation of carboxyl groups of glutamic and aspartic acid residues followed by the reaction with an amine group of another polypeptide chain to give an amide bond. Glutaraldehyde (GA) crosslinking of collagen provides materials with a high degree of crosslinking. Glutaraldehyde is a dialdehyde. The aldehyde is able to chemically interact with amino groups on collagen to form chemical bonds. This crosslinking agent is readily available, inexpensive, and forms aqueous solutions that can effectively crosslink tissue in a relatively short period. Using GA crosslinking, increased resistance to biodegradation and improved mechanical properties of collagen-based materials can be achieved. However, crosslinking of collagen-based materials using GA has shown to have cytotoxic characteristics, both in vitro and in vivo. Also, crosslinking of collagen-based materials using GA tends to result in stiffening of the material and calcification.

Crosslinking can also be accomplished with diisocyanates by bridging of amine groups on two adjacent polypeptide chains. In the first step, reaction of the isocyanate group with a (hydroxy)lysine amine group occurs, resulting in the formation of a urea bond. Thereafter a crosslink is formed by reaction of the second isocyanate group with another amine group. Diisocyanates do not show condensation reactions as observed in GA crosslinking. Also, no residual reagents are left in the material. A disadvantage, however, is the toxicity of diisocyanates and limited water solubility of most diisocyanates.

Yet another crosslinking method uses epoxy compounds to crosslink collagen. Epoxy compounds (i.e., epoxides) can undergo both acid-catalyzed and base-catalyzed reactions with a number of functional groups, including amine groups and carboxylic acid groups, under the appropriate conditions. However, it has now been noted that epoxy crosslinking agents introduce immunogenicity to the collagen-based material as well.

Additionally, current surgical techniques include endoscopic placement of collagen-based materials in an attempt help promote healing of, for example, a tendon. However, problems with all the foregoing collagen structures include a limited tensile strength, however. This is especially a factor at the materials fixation point to bone and other tendinous structures. Thus, improved implant materials and devices are needed.

Several embodiments disclosed herein solve one or more of these problems or others. Several embodiments disclosed herein provide orthopedic devices with better biological tolerance than those currently known. In several embodiments, disclosed herein are embodiments of crosslinked collagen-based materials. In several embodiments, devices (e.g., medical devices) comprising a crosslinked collagen-based material are provided (e.g., a crosslinked collagen-based cover). In several embodiments, the collagen-based material is crosslinked using an epoxide-based crosslinking agent. In several embodiments, the toxicity of the crosslinked collagen-based material is reduced by quenching any residual crosslinking agent in the collagen matrix using a quenching agent. Several embodiments disclosed herein also address issues involving the structural integrity of devices comprising crosslinked collagen-based materials. In several embodiments, for example, a device as disclosed herein may include an underlying support structure that provides additional structural integrity to an orthopedic as disclosed herein. Several embodiments of devices disclosed herein provide orthopedic implants with improved structural integrity and biocompatibility providing health benefits to patients in need of treatment.

### Devices

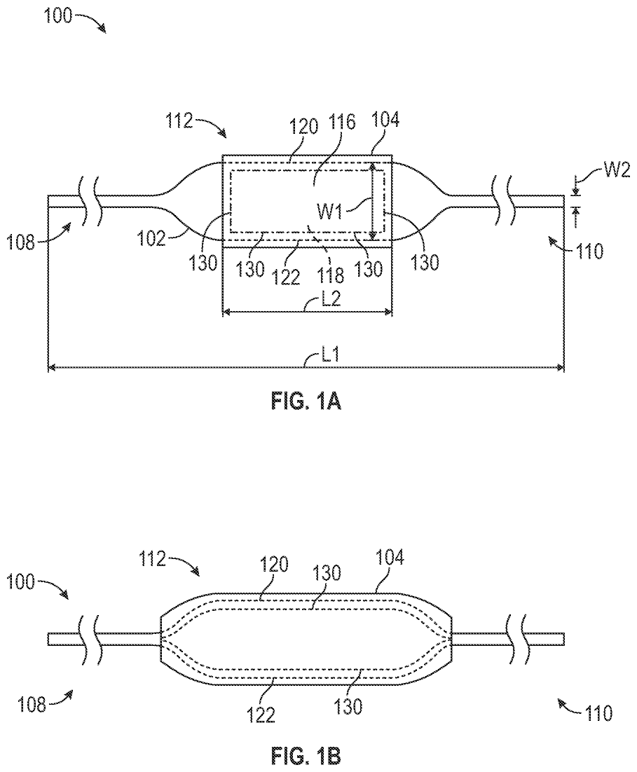

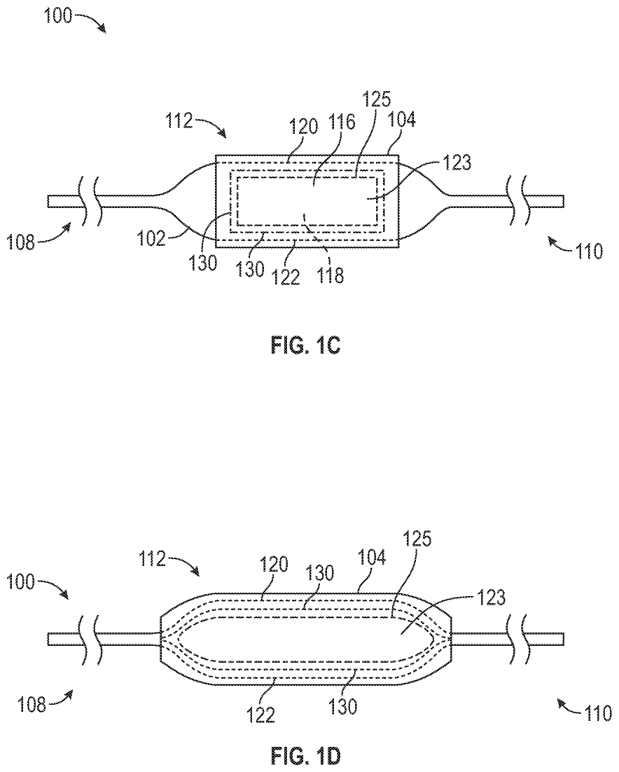

Some embodiments provide an orthopedic implant comprising a crosslinked collagen-based material (e.g., a crosslinked collagen-based covering). With reference to FIG. 1, some embodiments of the orthopedic implant 100 disclosed herein can have a support element 102 and a crosslinked collagen-based covering (also referred to herein as a covering or cover) 104 that can be attached to or can cover at least a portion of the support element 102.

Any of the embodiments of the orthopedic implants 100 disclosed herein can be configured for tendon, ligament, and/or other tissue repair, support, reconstruction and/or other treatment in any joint in the body. Embodiments of the orthopedic implants 100 disclosed herein can be particularly adapted for use in distal extremities. As used herein, the terms “distal extremity joint” and “distal extremity” are meant to refer to a joint or joints and other portions of the upper extremities and the lower extremities that are not connected to a body's trunk. Distal extremity joints of the upper extremities include all joints distal to and excluding the shoulder joint. The following are non-limiting examples of distal extremity joints of the upper extremities: ankle joints, wrist joints, elbow joints, hand joints, and finger or knuckle joints. Distal extremity joints of the lower extremities include all joints distal to and excluding the hip joint. The following are non-limiting examples of distal extremity joints of the lower extremities: knee joints, ankle joints, heel joints, foot joints, and toe or toe knuckle joints. Therefore, any of the embodiments disclosed herein configured for surgical repair, support, reconstruction and/or other treatment of the distal lower extremities can include orthopedic implants 100 configured for any of the following nonlimiting examples: ACL repair or treatment, for use in a Brostrom procedure for lateral ankle ligament reconstruction surgery that can be used to tighten up or firm up one or more ankle ligaments on an outside of the ankle, for Achilles tendon repair or treatment, and/or for repair or treatment of any tendons, ligaments, or other tissue in a patient's foot. Other embodiments disclosed herein can be configured for tendon, ligament, and/or other tissue repair, support, reconstruction and/or other treatment of the hip joint, for muscular and/or other tissue support, repair, treatment, and/or reconstruction of the abdominal wall, such as in a hernia treatment procedure, and/or for support, repair, treatment, and/or reconstruction of any of a patient's cervical joint(s). In some embodiments, the implant 100 can be configured to cover a joint or a sliding tendinous structure.

In any embodiments, the orthopedic implant 100 can be configured to provide support, reinforcement, tendon replacement, and/or other tissue replacement in any desired area of the body, for example and without limitation, in any distal extremity joint or distal extremity joints (as defined herein) of the body. As discussed, because patients can experience significant inflammation, irritation, pain, and/or discomfort with even minimal exposure to fabric and/or non-biological materials, any embodiments of the orthopedic implant 100 disclosed herein can be configured to eliminate, reduce, or inhibit inflammation and other negative side effects of exposure to the fabric and/or non-biological materials typically used for support elements in the joint by completely or substantially completely covering the portion of the support element or support elements that support the crosslinked collagen-based covering 104.

In any embodiments disclosed herein, the orthopedic implant 100 can be configured such that the crosslinked collagen-based covering 104 completely covers all surfaces of the support element 102 that extend over a length of the crosslinked collagen-based covering 104 (for example, all surfaces of the support element 102 except the portions of the support element 102 that extend past the crosslinked collagen-based covering 104). In several embodiments, the orthopedic implant can be configured such that the crosslinked collagen-based covering covers a majority of the surfaces or substantially covers all the surfaces of the support element that extend over a length of the crosslinked collagen-based covering. Some embodiments of the orthopedic implant 100 can have a crosslinked collagen-based covering 104 that extends over a first side of the support element 102, is folded over, and returns back so as to extend over the outer edge or side of the support element 102 and at least a second side of the support element 102, wherein the second side of the support element 102 is opposite to the first side of the support element 102. In this configuration, having the crosslinked collagen-based covering 104 extend continuously over the first side, outer edge or side, and at least second side of the support element 102 prevents exposure of the outer edge of the support element 102 from the anatomy surrounding or adjacent to the orthopedic implant 100, which can reduce or eliminate inflammation, irritation, pain, and/or discomfort to the tissue that surrounds or is adjacent to the support element 102, particularly the outer edge of the support element 102.

Additionally, some embodiments of the orthopedic implant 100 can have a crosslinked collagen-based covering 104 that extends over a first side of the support element 102, is folded over, and returns back so as to extend over the outer edge or side of the support element 102, at least a second side of the support element 102, and an inner edge or side of the support element 102 so as to completely cover the portion of the support element 102 or support elements 102 that extend over or adjacent to the crosslinked collagen-based covering 104. For example and without limitation, the inner edge or side of the support element 102 can be covered by the crosslinked collagen-based covering 104 and, in some embodiments, one or more stitches can extend along a length of the crosslinked collagen-based covering 104 that can essentially seal off or partially seal off the return edge of the crosslinked collagen-based covering 104 and the inner edge of the support element 102 that is covered by the crosslinked collagen-based covering 104.

In some embodiments, a baseball stitching pattern, a herringbone stitching pattern, or other suitable stitching pattern can be used to essentially seal off or partially seal off the edge of the crosslinked collagen-based covering 104 and the inner edge of the support element 102 that is covered by the crosslinked collagen-based covering 104. Such stitches can have a first portion that engages both layers of the crosslinked collagen-based covering 104 only and a second portion that engages both layers of the crosslinked collagen-based covering 104 and the support element 102. The individual stitches can be at an angle that extends over the inner edge or side of the support element 102.

The crosslinked collagen-based material can be folded and sewn to cover at least a portion of the support element 102. In any embodiments disclosed herein, the support element 102 can be made from one or more strands or sutures of a biocompatible polymeric material or other suitable material that are braided. For example and without limitation, some embodiments of the support element 102 can include braided sutures made from polyethylene and/or ultra-high molecular weight polyethylene (UHMWPE), polyetheretherketone (PEEK), and/or any other suitable or desired materials. The support element 102 of any embodiments disclosed herein can include one or more sutures made from a multi-stranded, long chain UHMWPE core with a braided jacket of polyester and UHMWPE. In any embodiments disclosed herein, the support element 102 can be made from a sheet of material, such as polytetrafluoroethylene (PTFE) or other suitable or desired material. The sheet of material can be die cut to the desired shape of the support element 102, or otherwise formed to the desired shape of the support element 102.

In some embodiments, sutures can be sewn through one or both layers of the crosslinked collagen-based covering 104 (i.e., both layers of the crosslinked collagen-based covering 104 that have been folded over the support element 102) and the support element 102 on each side of the crosslinked collagen-based covering 104 to secure the support element 102 to each side of the crosslinked collagen-based covering 104. In this configuration, the support element 102 can be secured to the crosslinked collagen-based covering 104 in an axial direction of the support element 102 also to prevent, for example and without limitation, the support element 102 from sliding relative to the crosslinked collagen-based covering 104. One or two, or more, rows of sutures can pass through the support element 102 and crosslinked collagen-based covering 104 to secure the support element 102 to each side of the crosslinked collagen-based covering 104. The sutures can be arranged in a linear pattern, a zig-zag pattern, or in any other suitable pattern. In other embodiments, one or more sutures can be positioned along one or both sides of the support element 102 and pass through both layers of the crosslinked collagen-based covering 104 without passing through the support element 102. In some embodiments, this can be done to allow the support element 102 to be slideable relative to the crosslinked collagen-based covering 104.

In some embodiments, sutures can be sewn through one or both layers of the crosslinked collagen-based covering 104 (i.e., both layers of the crosslinked collagen-based covering 104 that have been folded over the support element 102) and the support element 102 (which can be, without limitation, a surgical mesh tape) on each side of the crosslinked collagen-based covering 104 to secure the support element 102 to each side of the crosslinked collagen-based covering 104. In this configuration, the support element 102 can be secured to the crosslinked collagen-based covering 104 in an axial direction of the support element 102 also to prevent, for example and without limitation, the support element 102 from sliding relative to the crosslinked collagen-based covering 104. One or two, or more, rows of sutures can pass through the support element 102 and crosslinked collagen-based covering 104 to secure the support element 102 to each side of the crosslinked collagen-based covering 104. The sutures can be arranged in a linear pattern, a zig-zag pattern, or in any other suitable pattern. In other embodiments, one or more sutures can be positioned along one or both sides of the support element 102 and pass through both layers of the crosslinked collagen-based covering 104 without passing through the support element 102. In some embodiments, this can be done to allow the support element 102 to be slideable relative to the crosslinked collagen-based covering 104.

Some embodiments of the support element 102 can have a first end portion 108, a second portion 110, and a middle portion 112 between the first end portion 108 and the second end portion 110. In some embodiments, the middle portion 112 can have a first main surface 116 having a first width W1, a second main surface 118 opposite to the first main surface 116, a first side edge surface 120, and a second side edge surface 122. In any embodiments disclosed herein, the first and second main surfaces 116, 118 can be flat or planar. The first width W1 can extend from the first side edge surface 120 to the second side edge surface 122. In some embodiments, the first and second end portions 108, 110 can have a width W2 that is less than the first width W1 of the middle portion 112 or, put another way, the first width W1 of the middle portion 112 of any embodiments disclosed herein can be greater than the second width W2 of the first end portion 108 and/or the second end portion 110. For example and without limitation, the first width W1 of the middle portion 112 of any embodiments of the support element 102 disclosed herein can be 100%, or approximately 100%, or from 80% to 120% greater than the second width of the first end portion 108 and/or the second end portion 110 of the support element 102 (i.e., the first width W1 of the middle portion 112 can be twice as wide or approximately twice as wide as the first end portion 108 and/or the second end portion 110 of the support element 102, or can be from 40% (or approximately 40%, or less than 40%) to 500% (or approximately 500%, or more than 500%) greater than the second width W2 of the first end portion 108 and/or the second end portion 110 of the support element 102, or can be from 100% (or approximately 100%) to approximately 300% (or approximately 300%) greater than the second width W2 of the first end portion 108 and/or the second end portion 110 of the support element 102, or greater than the second width W2 of the first end portion 108 and/or the second end portion 110 of the support element 102 by any of the values within any of the foregoing ranges, or from and to any of the values within any of the foregoing ranges. Further, in some embodiments, the first width W1 of the middle portion 112 of the support element 102 can be the same as or approximately the same as the second width of the first end portion 108 and/or the second end portion 110 of the support element 102. In any embodiments disclosed herein, the first width W1 and/or a length of the middle portion 112 can be greater than a thickness of the middle portion 112 or at least the middle portion 112.

In some embodiments, the crosslinked collagen-based covering 104 only covers the middle portion 112 and does not cover the first end portion 108 or the second end portion 110 of the support element 102. This can permit the first end portion 108 and the second end portion 110 to maintain a smaller profile that can more easily be drawn through anchors to secure the orthopedic implant 100 having the crosslinked collagen-based covering 102 in the desired anatomical location. In some embodiments, the crosslinked collagen-based covering 104 can cover at least the first main surface 116, the second main surface 118, and the first side edge. In some embodiments, the crosslinked collagen-based covering 104 can be wrapped around at least the middle portion 112 of the support element 102.

With reference to FIGS. 1C and 1D, any embodiments of the support element 102 disclosed herein can have an opening or cutout 123 in at least a middle portion 112 of the support element 102. In some embodiments, the opening 123 can have perimeter edges 125 of any desired shape, including an oval shape, an elliptical shape, an elongated shape, a circular shape, or otherwise. In some embodiments, the shape of the opening 123 can generally match a shape of an outside perimeter of the support element 102 adjacent to the opening 123. The crosslinked collagen-based covering 104 can extend over the opening 123 such that the entire opening 123 and the portions of the support element 102 adjacent to the opening 123 are covered by the crosslinked collagen-based covering 104. In the region of the opening, some embodiments of the implant device 100 can be configured to have only the crosslinked collagen-based covering 104. One or more sutures 130 can be passed through the support element 102 and the crosslinked collagen-based covering 104 adjacent to the opening 123 to secure the crosslinked collagen-based covering 104 to the support element 102 and/or prevent or inhibit the portions of the support element 102 adjacent to the opening 123 from changing shape or moving relative to the crosslinked collagen-based covering 104.

Embodiments of the implant device 100 having the opening 123 in the support element 102 can be used in regions of the body or applications where it is beneficial to have the crosslinked collagen-based covering 104 for in-growth, for example and without limitation, without an underlying braided support element material. This configuration of the implant 100 having the opening 123 in the support element 102 can also maximize the area of the crosslinked collagen-based covering 104 without increasing a cross-sectional area of the support element 102.

Embodiments of the implant device 100 having the opening 123 in the support element 102 can be used in any distal extremity joint in the body, in the hip, for knee cap surgical procedures, or for other applications as disclosed herein.

In some embodiments, the support element 102 can be braided so that the opening 123 is formed during the braiding process. This can have the advantage of ensuring that there are no cut ends of the sutures or other materials used for the braid between the first and second end portions 108, 110. In other embodiments, the opening 123 can be formed in the middle portion 112 of the support element 102 after the braiding process, for example and without limitation, using die cut tooling or other cutting process.

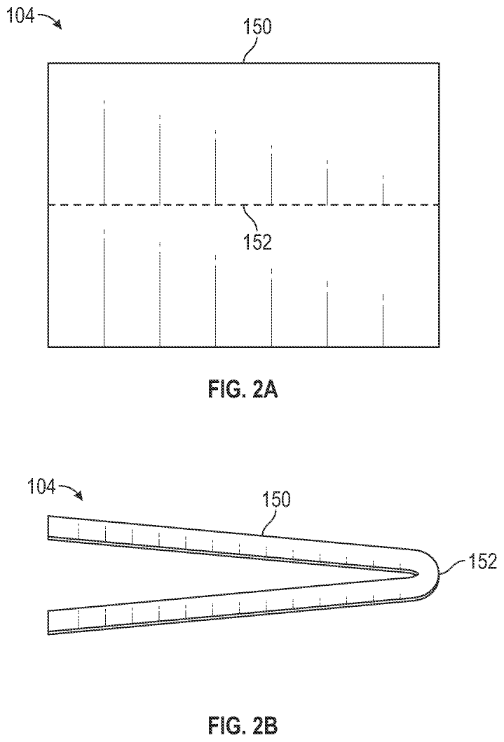

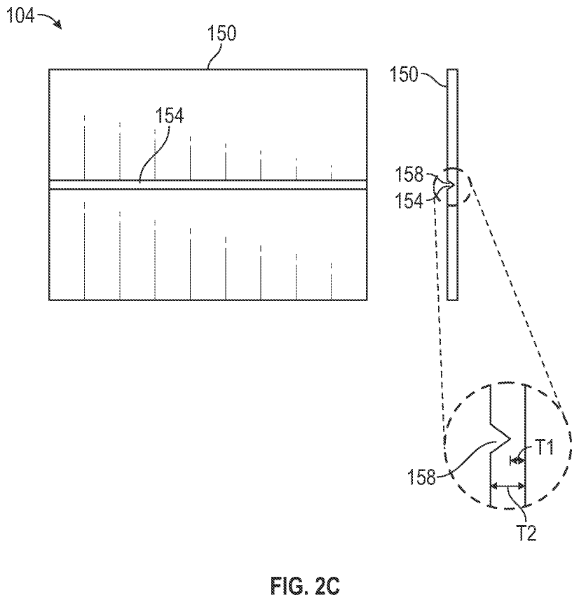

For example and without limitation and as shown in FIG. 2A, the crosslinked collagen-based covering 104 can include a sheet 150 of the collagenous material (also referred to herein as a collagenous substrate) that can be wrapped around at least the first main surface 116, the second main surface 118, and the first side edge surface 120 of the middle portion 112 of the support element 102. Any embodiments of the crosslinked collagen-based covering 104 can include a sheet 150 of the collagenous material that can be wrapped continuously around the first main surface 116, the second main surface 118, the first side edge surface 120, and the second side edge surface 122 of the middle portion 112 of the support element 102.

In some embodiments and as shown in FIGS. 2A-2B, though not required, the crosslinked collagen-based covering 104 can include a sheet 150 of the collagenous material. Some embodiments of the crosslinked collagen-based covering 104 or the sheet 150 can have at least one fold 152 along a length thereof. The fold 152 can be configured to facilitate the wrapping and/or bending of the sheet 150 of the collagenous material on itself (in embodiments where there is no additional layer or reinforcing material), and/or to facilitate the wrapping and/or bending of the sheet 150 of the collagenous material over and/or around a support element 102. One or more folds 152 can be positioned in a middle portion of the sheet 150, along a centerline of the sheet 150, adjacent to the middle portion of the sheet 150, or at any desired position or location on the sheet 150.

Some embodiments of the sheet 150 or other crosslinked collagen-based covering 104 can include two or more folds 152, for example and without limitation, two or more folds 152 can extend along a length of the sheet 150 and be oriented to align with the first side edge surface 120 and the second side edge surface 122 of the support element 102 so that the sheet 150 can be wrapped around the support element 102 and cover the first side edge surface 120 and the second side edge surface 122. In some embodiments, the one or more folds 152 can facilitate a more uniform height or thickness of the crosslinked collagen-based covering 104 along a length and over a width of the crosslinked collagen-based covering 104 and/or of the orthopedic implant 100 along the middle portion of the support element 102 or other portion of the orthopedic implant 100 covered by the crosslinked collagen-based covering 104.

In some embodiments, with reference to FIG. 2C, the crosslinked collagen-based covering 104 and/or sheet 150 of crosslinked collagen-based covering 104 can include one or more cuts 154 along a length thereof. In some embodiments, the one or more cuts 154 can coincide with the one or more folds 152 formed in the sheet 150 to further facilitate wrapping of the crosslinked collagen-based covering 104 around and/or over the support element 102, or to further facilitate the folding of the sheet 150 or the crosslinked collagen-based covering 104 on itself in embodiments that do not have the support element 102. In some embodiments, the at least one fold 152 can be aligned with the first side edge surface 120 of the support element 102. In some embodiments, the one or more cuts 154 can include or can form at least one channel 158 having a reduced thickness (represented by T1 in FIG. 2C) along the at least one channel 158. The reduced thickness T1 can be less than a thickness T2 of the crosslinked collagen-based covering 104 adjacent to the at least one channel 158. In some embodiments, the reduced thickness T1 of the channel 158 can be 0.1 mm or approximately 0.1 mm, or from 0.05 mm to 0.15 mm, and the thickness T2 of the crosslinked collagen-based covering 104 adjacent to the at least one channel 158 can be 0.5 mm or approximately 0.5 mm, or from 0.3 mm to 0.7 mm. In some embodiments, the reduced thickness T1 of the channel 158 can be from 10% to 50% of the thickness T2 of the crosslinked collagen-based covering 104 adjacent to the at least one channel 158, or from 15% to 30% of the thickness T2 of the crosslinked collagen-based covering 104 adjacent to the at least one channel 158, or 20% or approximately 20% of the thickness T2 of the crosslinked collagen-based covering 104 adjacent to the at least one channel 158.

In any embodiments, the crosslinked collagen-based covering 104 can be attached to the support element 102 using one or more sutures 130. Sutures can pass through the 102 or can be configured to pass only through the 104. The sutures 130 in any embodiments can pass through the support element 102 and/or the crosslinked collagen-based covering 104. For example, the sutures 130 can pass through both layers of the crosslinked collagen-based covering 104 outside of a periphery of the support element 102, for example, adjacent to one or more edges of the crosslinked collagen-based covering 104 spaced apart from or adjacent to the first side edges surface 120 and/or the second side edge surface 122. Some embodiments of the orthopedic implant 100 can have multiple lines of sutures extending along a length of the crosslinked collagen-based covering 104.

In any embodiments disclosed herein, the support element 102 can include at least one of a thread, a suture, a sheet, a strip, a fabric, a mesh material such as a polymeric surgical mesh material, and a weave. In some embodiments, the support element 102 can include a woven material that can be wider in the middle portion 112 than in the first or second end portions 108, 110. For example and without limitation, in some embodiments, the first width of the middle portion 112 of the support element 102 can be greater than a width of the first end portion 108 and/or the second end portion 110 of the support element 102. In some embodiments, support element 102 can have a length L1 from an end of the first end portion 108 to an end of the second end portion 110, and the crosslinked collagen-based covering 104 can have a length L2 parallel to the first side edge surface 120 and the second side edge surface 122. In some embodiments, the length L1 of the support element 102 can be greater than the length L2 of the crosslinked collagen-based covering 104. In some embodiments, the length L1 of the support element 102 can be 100% greater than (i.e., double) the length L2 of the crosslinked collagen-based covering 104, or can be 200% greater than (i.e., triple) the length L2 of the crosslinked collagen-based covering 104, or can be from 100% (or approximately 100%) greater than the length L2 of the crosslinked collagen-based covering 104 to 300% (or approximately 300%, or more than 300%) greater than the length L2 of the crosslinked collagen-based covering 104.

In some embodiments, the crosslinked collagen-based covering 104 can be configured to be cut in an anatomical shape to match a shape of a natural tendinous structure requiring surgical repair when attached to the support element 102. In some embodiments, the assembly/construction process of the orthopedic implant can be configured so as to minimize an exposure of the underlying support element 102. For example and without limitation, in some embodiments, the crosslinked collagen-based covering 104 can cover an entire periphery of the middle portion 112 of the support element 102 along a length of the support element 102 that is to be covered by the crosslinked collagen-based covering 104. In some embodiments, the orthopedic implant can be configured such that no portion of the support element 102 in the middle portion 112 of the support element 102 is exposed or is uncovered by the crosslinked collagen-based covering 104.

In other embodiments, the orthopedic implant can include a crosslinked collagen-based covering 104 or layer without having a support element or other reinforcing component or element, such as for low load applications. For example and without limitation, some embodiments of the orthopedic implant can have a crosslinked collagen-based covering 104 having any of the components, features, and/or other details of any of the crosslinked collagen-based substrates or material disclosed herein that can be configured for use in a repair procedure for any of the distal extremity joints. In some embodiments, sutures or other fastening or anchoring means can be used, but are not required to be used, such as in applications where it may be beneficial for the crosslinked collagen-based covering 104 to move or slide relative to the support element 102, to secure the crosslinked collagen-based covering 104 in the desired position. Additionally, some embodiments of the crosslinked collagen-based covering 104 can be used in hernia procedures or operations, the crosslinked collagen-based covering 104 in some embodiments being positioned over the defect to reinforce the abdominal wall. In some embodiments, the crosslinked collagen-based covering 104 can be positioned between muscle layers in a hernia procedure.

Any embodiments of the orthopedic implant 100 disclosed herein can be configured for treating a tissue defect, wherein the orthopedic implant 100 of any embodiments herein can be configured for positioning at, over, or into the tissue defect. In some embodiments, the tissue defect can be a wound. Some embodiments provide a method for performing tissue repair and/or for providing tissue and organ supplementation. A non-limiting list of applications for which any of the orthopedic implant 100 embodiments disclosed herein can be configured includes diabetic foot ulcers, venous leg ulcers, pressure ulcers, amputation sites, wounds, and/or other skin trauma or ailments.

Some embodiments of the orthopedic implant 100 disclosed herein can be secured to the desired joint or location using one, two, three or more anchors. The anchor or anchors can be implanted into or otherwise secured to the boney structures of or around the joint. In some embodiments, each of the first and/or second ends 108, 110 can be passed through an eyelet in the anchor or otherwise coupled with an anchor that is secured to the boney structure of or around the joint. The anchors can be implanted in any desired manner, including without limitation, drilling a bore hole and threadedly inserting the anchor. In some embodiments, the bore hole can be tapped or threaded prior to threadedly inserted in the anchor. Anchors, sutures, hooks, barbs, and/or adhesive can be used to secure or couple any of the embodiments of the orthopedic implants disclosed herein to a patient's distal extremity joint or other anatomy disclosed herein. In other embodiments, spring wire or memory wire constructs, or other constructs made from polymeric materials and/or metal can be used to secure or couple any of the embodiments of the orthopedic implants disclosed herein to a patient's distal extremity joint or other anatomy disclosed herein, alone or in combination with adhesives.

### Compositions

As disclosed elsewhere herein, in several embodiments, an orthopedic implant as disclosed herein may comprise a crosslinked collagen-based material. In several embodiments, the crosslinked collagen-based material may be molded or cut into a suitable shape to act as a covering for an orthopedic implant (e.g., to partially, substantially, or fully cover an orthopedic implant, a suture, etc.). In several embodiments, the crosslinked collagen-based material comprises a collagenous substrate. In several embodiments, the collagenous substrate comprises collagen strands. In several embodiments, one or more collagen strands are linked together (e.g., crosslinked) by a species that spans from a first collagen strand to a second collagen strand. For example, a crosslinking agent can be used to form a crosslink that spans two collagen strands. In several embodiments, the collagenous substrate comprises a matrix of collagen strands. In several embodiments, the collagen strands (e.g., of the matrix) are crosslinked by one or more crosslinking units that bridge the strands. In several embodiments, each crosslinking unit is derived from a crosslinking agent (e.g., is formed after the reaction of a crosslinking agent with reactive groups of collagen strands during synthesis of the crosslinked collagen-based material). In several embodiments, when two or more reactive groups of a crosslinking agent react with two or more collagen strands, a crosslink between those collagen strands is provided (thereby providing the crosslinking unit).

In several embodiments, the crosslinked collagen-based material is a treated crosslinked collagen-based material. For example, as noted elsewhere herein, it has now been noted that using a quenching agent consumes residual unreacted crosslinking agent within the crosslinked collagen-based material, thereby lowering toxicity of the resultant treated crosslinked collagen-based material (versus a crosslinked collagen-based material that has not be quenched). In several embodiments, a collagen-based material of the orthopedic device comprises, consists of, or consists essentially of treated crosslinked collagen-based material.

In several embodiments, the crosslinking agent comprises a multifunctional epoxide. A multifunctional epoxide is a molecule that comprises a plurality of epoxides functional groups (e.g., 2, 3, 4, or more). In several embodiments, the multifunctional epoxide is a diepoxide. As used herein, the term “diepoxide” refers to a compound that has two reactive epoxide functionalities. Useful diepoxides may include, but are not limited to, glycol diglycidyl ether, glycerol diglycidyl ether, butanediol diglycidyl ether, resorcinol diglycidyl ether, 1,6-hexanediol diglycidyl ether, ethylene glycol diglycidyl ether, diethylene glycol diglycidyl ether, triethylene glycol diglycidyl ether, polyethylene glycol diglycidyl ether, propylene glycol diglycidyl ether, dipropylene glycol diglycidyl ether, and polybutadiene, diglycidyl ether. An example of the diepoxide that can be used to crosslink collagen strands is 1,4 butanediol digylcidyl ether (BDDGE). Multifunctional epoxides may include, but are not limited to, the above mentioned diepoxides, glycerol triglycidyl ether, sorbitol polyglycidyl ether, polyglycerol polyglycidyl ether, pentaerythritol polyglycidyl ether, diglycerol polyglycidyl ether, glycerol polyglycidyl ether, and trimethylolpropane polyglycidyl ether.

In several embodiments, the crosslinking agent comprises a structure represented by Formula (A):

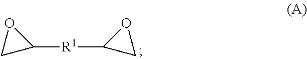

where R¹ as disclosed elsewhere herein. In several embodiments, R¹ is selected from the group consisting of optionally substituted alkylene, optionally substituted polyether, and optionally substituted polyamino. In several embodiments, R¹ is selected from the group consisting of optionally substituted C₁₋₆ alkylene, —R_{E}—(N(R_{A})R_{B}—)ₙ—N(R_{C})—R_{F}—, and —(R_{E})—(OR_{B}—)ₙO—R_{F}—, where the variables are as disclosed elsewhere herein. In several embodiments, R¹ is represented by a structure selected from the group consisting of: —(CH₂)ₐ—(O—(CH₂)_{b})_{c}—O—(CH₂)_{d}—, —(CH₂)ₐ—(NH—(CH₂)_{b})_{c}—NH—(CH₂)_{d}—, and —(CH₂)ₐ—. In several embodiments, each of a, b, c, and d is independently an integer equal to 0, 1, 2, 3, 4, 5, 6, 7, or 8. In several embodiments, R¹ is represented by —CH₂—O—(CH₂)_{b}—O—CH₂— and b is 4.

In several embodiments, after crosslinking of the collagen matrix, the crosslinked collagen-based material comprises a crosslinking unit. In several embodiments, the crosslinking unit is formed through reaction with a first amine of the collagen matrix and a second amine of the collagen matrix. In several embodiments, the first amine is part of a first collagen strand of the collagenous substrate and the second amine is part of a second collagen strand of the collagenous substrate. In several embodiments, the crosslink is represented by Formula (I):

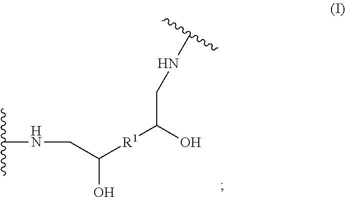

In several embodiments, R¹ is selected from the group consisting of optionally substituted alkylene, optionally substituted polyether, and optionally substituted polyamino. In several embodiments, R¹ is selected from the group consisting of optionally substituted C₁₋₆ alkylene, —R_{E}—(N(R_{A})R_{B}—)ₙ—N(R_{C})—R_{F}—, and —(R_{E})—(OR_{B}—)ₙO—R_{F}—, where the variables are as disclosed elsewhere herein. In several embodiments, R¹ is represented by a structure selected from the group consisting of: —(CH₂)ₐ—(O—(CH₂)_{b})_{c}—O—(CH₂)_{d}—, —(CH₂)ₐ—(NH—(CH₂)_{b})_{c}—NH—(CH₂)_{d}—, and —(CH₂)ₐ—. In several embodiments, each of a, b, c, and d is independently an integer equal to 0, 1, 2, 3, 4, 5, 6, 7, or 8. In several embodiments, R¹ is represented by —CH₂—O—(CH₂)_{b}—O—CH₂— and b is 4.

In several embodiments, the crosslink of Formula (I) is further represented by Formula (Ia):

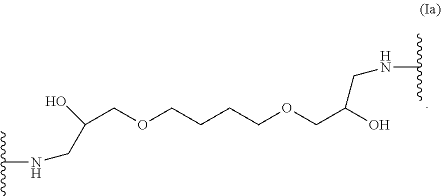 [SMILES: *NCC(O)COCCCCOCC(O)CN*]

In several embodiments, the crosslinking unit is formed through reaction with a carboxylic acid of the collagen matrix and an amine of the collagen matrix. In several embodiments, the carboxylic acid is part of a first collagen strand of the collagenous substrate and the amine is part of a second collagen strand of the collagenous substrate. In several embodiments, the crosslink is represented by Formula (Ic):

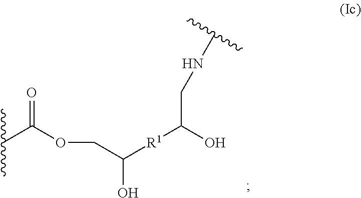

where R¹ is as disclosed elsewhere herein. In several embodiments, the variables defined for one structural formula may also be used to define that variable in any other formula having that same variable. For example, when a variable has the same alphanumeric designation (e.g., R¹) for one formula (e.g., Formula (I)), that definition of the variable can be used in other formulae (e.g., Formulae (Ic) or (Ia)), even where the variable is not specifically defined for those other formulae.

In several embodiments, the crosslinking unit is formed through reaction with a first carboxylic acid of the collagen matrix and a second carboxylic acid of the collagen matrix. In several embodiments, the first carboxylic acid is part of a first collagen strand of the collagenous substrate and the second carboxylic acid is part of a second collagen strand of the collagenous substrate. In several embodiments, the crosslink is represented by Formula (Id):

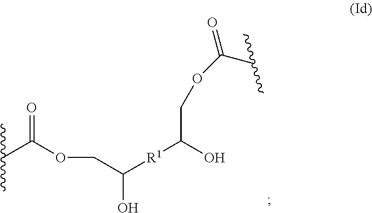

where R¹ is as disclosed elsewhere herein.

As disclosed elsewhere herein, multifunctional epoxides have been used as crosslinking agents for collagen to improve physical properties of the material. However, it has now been noted that using multifunctional epoxides may lead to immunogenicity (e.g., cytotoxicity, etc.) of the crosslinked collagen material. It has now been noted that, when using epoxide crosslinking agents, epoxides of the crosslinking agent may not fully react, leaving residual epoxides present in the material. This reactive group (e.g., a residual epoxide) can be toxic and/or immunogenic. To illustrate, a crosslinked collagen material may comprise both Formula (I), (Ia), (Ic), and/or (Id) above, but at the same time may comprise units represented by Formula (Ib):

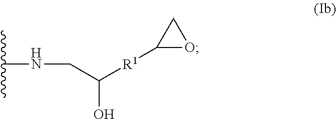

where R¹ is as described elsewhere herein. Additionally, a crosslinked collagen material may comprise both Formula (I), (Ia), (Ib), (Ic), and/or (Id) above, but at the same time may comprise units represented by Formula (Ie):

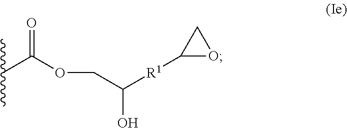

where R¹ is as described elsewhere herein.

In several embodiments, it has now been noted that using a quenching agent consumes residual epoxides, thereby lowering toxicity of the crosslinked collagen-based material. For example, a nucleophilic small molecule may be used to react with the residual epoxide groups (such as the residual epoxide shown in Formulae (Ib) and/or (Ie)). In several embodiments, the quenching group may be represented by the following formula H—X¹—R² where X¹ is a nucleophilic group. In several embodiments, X¹ is selected from the group consisting of —O— and —N(R₃)—, where R³ is selected from the group consisting of —H and optionally substituted C₁₋₆ alkyl. In several embodiments, X¹ is selected from the group consisting of —O— and —NH—. In several embodiments, R² is selected from the group consisting of optionally substituted alkyl, optionally substituted polyether, and optionally substituted polyamino. In several embodiments, R² is optionally substituted C₁₋₆ alkyl. In several embodiments, R² is —CH₂CH₃.

As disclosed elsewhere herein, any one or more of R¹, R², and R³ may be optionally substituted. In several embodiments, an optional substitution may be as disclosed above. In several embodiments, when an R¹ group is substituted with one or more optional substitutions, the one or more optional substitutions are independently selected from the group consisting of acyl, C₁₋₆ alkyl, C₁₋₆ alkoxy, C-amido, halogen, and hydroxy. In several embodiments, when an R² group is substituted with one or more optional substitutions, the one or more optional substitutions are independently selected from the group consisting of acyl, C₁₋₆ alkyl, C₁₋₆ alkoxy, C-amido, halogen, and hydroxy. In several embodiments, when an R³ group is substituted with one or more optional substitutions, the one or more optional substitutions are independently selected from the group consisting of acyl, C₁₋₆ alkyl, C₁₋₆ alkoxy, C-amido, halogen, and hydroxy.

In several embodiments, the crosslinked collagen-based material comprises a quenched crosslinking agent (e.g., by performing a quench using a quenching group as disclosed elsewhere herein). In several embodiments, the quenched crosslinking agent is bonded to the collagenous material an amine (e.g., a third amine) of the collagenous substrate. In several embodiments, the crosslinked collagen-based material comprises a structure represented by Formula (II):

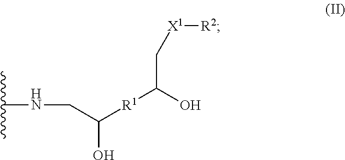

where R² and X¹ are as disclosed elsewhere herein. In several embodiments, R² is selected from the group consisting of optionally substituted alkyl, optionally substituted polyether, and optionally substituted polyamino. In several embodiments, X¹ is selected from the group consisting of —O— and —NH—. In several embodiments, R² is represented by a structure selected from the group consisting of: —(CH₂)ₐ—(O—(CH₂)_{b})_{c}—O—(CH₂)_{d}—H, —(CH₂)ₐ—(NH—(CH₂)_{b})_{c}—NH—(CH₂)_{d}—H, and —(CH₂)ₐ—H, where each of a, b, c, and d is independently an integer equal to 0, 1, 2, 3, 4, 5, 6, 7, or 8. In several embodiments, Formula (II) is further represented by Formula (IIa):

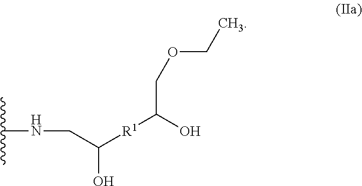

In several embodiments, the crosslinked collagen-based material comprises a structure represented by Formula (IIb):

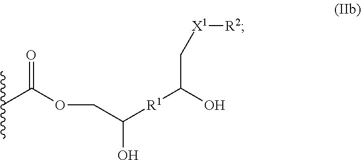

where R² and X¹ are as disclosed elsewhere herein.

In several embodiments, the crosslinked collagen-based material comprises a structure represented by any one or more of Formula (I), Formula (Ia), Formula (Ic), Formula (Id), Formula (II), Formula (IIa), Formula (IIb), and/or combinations of any of the foregoing. In several embodiments, the crosslinked collagen-based material comprises a structure represented by any one or more of Formula (I), Formula (Ia), Formula (Ic), Formula (Id), Formula (II), Formula (IIa), Formula (IIb), and/or combinations of any of the foregoing, but lacks, substantially lacks, or has a reduced amount of Formula (Ib) and/or (Ie). For example, in several embodiments, the crosslinked collagen-based material comprises a quenched crosslinking agent (e.g., prepared by performing a quench using a quenching group as disclosed elsewhere herein). In several embodiments, the quenched crosslinking agent is bonded to the collagenous material via an amine (e.g., a third amine) of the collagenous substrate. In several embodiments, the quenched crosslinking agent is bonded to the collagenous material via carboxylic acid (e.g., a third carboxylic acid) of the collagenous substrate.

In several embodiments, each instance of “ [symbol] ” of Formula (I), Formula (Ia), Formula (Ib), Formula (Ic), Formula (Id), Formula (Id), Formula (II), Formula (Ha), and/or Formula (IIb) represents a portion of the collagenous substrate (e.g., a collagen strand of the collagen matrix).

In several embodiments, the crosslinked collagen-based material has desirable material properties. In several embodiments, the crosslinked collagen-based material has improved properties relative to materials that are not quenched. In several embodiments, the treated (e.g., quenched) crosslinked collagen-based material has a shrinkage temperature (Ts) equal to or at least about: 70° C., 75° C., 76° C., 77° C., 78° C., 70° C., 70° C., or ranges including and/or spanning the aforementioned values. In several embodiments, the treated crosslinked collagen-based material has a tensile strength equal to or at least about: 5 N, 7.5 N, 8.0 N, 9.0 N, 10 N, 11 N, 12 N, 15N, or ranges including and/or spanning the aforementioned values. Tensile strength can be measured using tensile bars (40.0 mm×4.0 mm×1.4 mm) cut using a dumb-bell shaped knife and can be hydrated for at least one hour in PBS at room temperature. The thickness of the samples can be measured in triplicate using a spring-loaded type micrometer. An initial gauge length of 10 mm can be used and a crosshead speed of 5 mm/minute can be applied until rupture of the test specimen occurs. A preload of 0.05 N can be applied to pre-stretch the specimen before the real measurement. In several embodiments, the treated crosslinked collagen-based material retains properties (e.g., tensile strength and/or shrinkage temperature) for a surprisingly long period of time, for example, decreasing less than 10% after a period of equal to or greater than about: 1 month, 2 months, 3 months, 4 months, 6 months, 1 year, or ranges including and/or spanning the aforementioned values.

In several embodiments, the treated crosslinked collagen-based material is tailored to have a variable degradation rate depending on the application of the orthopedic implant. In several embodiments, the degradation rate of the treated crosslinked collagen-based material may be measured using HEPES buffered solution with a concentration of 95 mg/100 ml bacterial protease derived from Streptomyces griseus and an incubation time at 45° C. of 24 hours. In several embodiments, the degradation rate of the treated crosslinked collagen-based material may be measured using a pronase digestion assay. In several embodiments, the treated crosslinked collagen-based material has a degradation rate (in a pronase digestion assay as disclosed herein) per hour of less than or equal to about: 0.01%, 0.1%, 0.2%, 0.3%, 0.5%, 0.75%, 1.0%, 2.0%, or ranges including and/or spanning the aforementioned values. In several embodiments, the treated crosslinked collagen-based material has a degradation rate of between 0.2% or about 0.2% to 1.0% or about 1.0% per hour when measured using the pronase digestion assay described in the EXAMPLES section. In some embodiments, the treated crosslinked collagen-based material has a degradation rate (in a pronase digestion assay as disclose herein) that ranges from 0.1% to 1.10% (or about 0.1% to about 1.10%) per hour, from 0.3% to 1.0% (or about 0.3% to about 1.0%) per hour, from 0.4% to 0.9% (or about 0.4% to about 0.9%) per hour, from 0.5% to 0.8% (or about 0.5% to about 0.8%) per hour, from 0.6% to 0.7% (or about 0.6% to about 0.7%) per hour, from 0.2% to 0.3% (or about 0.2% to about 0.3%) per hour, from 0.3% to 0.4% (or about 0.3% to about 0.4%) per hour, from 0.4% to 0.5% (or about 0.4% to about 0.5%) per hour, from 0.5% to 0.6% (or about 0.5% to about 0.6%) per hour, from 0.6% to 0.7% (or about 0.6% to about 0.7% per hour), from 0.7% to 0.8% (or about 0.7% to about 0.8%) per hour, from 0.8% to 0.9% (or about 0.8% to about 0.9%) per hour, from 0.9% to 1.0% (or about 0.9% to about 1.0% per hour), or from 1.0% to 1.1% (or about 1.0% to about 1.1% per hour).

### Methods of Manufacture

Some embodiments provide a method of making a treated crosslinked collagen-based material. In several embodiments, the method comprises providing a collagen material. In several embodiments, the collagen material is exposed to a crosslinking solution comprising a crosslinking agent to provide a crosslinked collagen-based material. In several embodiments, the crosslinked collagen-based material is exposed to a quenching agent to provide the treated crosslinked collagen-based material. Several embodiments pertain to making an orthopedic implant by shaping the treated crosslinked collagen-based material to provide a portion of the orthopedic implant.

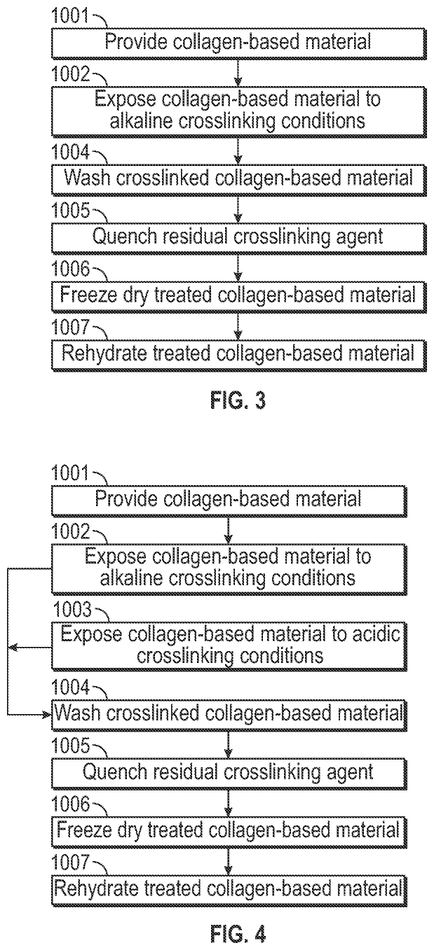

In several embodiments, as shown in FIGS. 3 and 4, the method may include a first step (101) that includes providing a collagen-based material. In some embodiments, animal or human tissue is dissected and undergoes a decellularization process to result in the collagen-based material. In some embodiments, depending on the level of processing of natural tissue, collagen-based materials may be collagen, tropocollagen, collagen fibrils, or collagen fibers. In some embodiments, the collagen-based material is excised from the pericardium of an animal or a human. In several embodiments, collagen tissue is trimmed, cleaned of fat, debris and blood in a saline rinse. In several embodiments, the tissue is de-cellularized using sonication with an anionic surfactant (Sodium Dodecyl Sulfate) to remove a majority of intracellular materials. In several embodiments, a collagen source may include pericardium from a bovine, equine, human, or other source.

In several embodiments, as shown in FIGS. 3 and 4, the collagen-based material is exposed to solution comprising a crosslinking agent in a crosslinking step (1002). In several embodiments, the solution is a buffered solution. In several embodiments, as shown in FIGS. 3 and 4, the crosslinking step may be performed using a solution with an alkaline pH. In several embodiments, the pH is high enough to result in crosslinks that are primarily and/or exclusively amine-based. In several embodiments, the solution (e.g., buffered solution) has a pH of greater than or equal to 8.0, 8.5, 9.0, 9.2, 9.5, 10.0, 10.5, 11.0, or ranges including and/or spanning the aforementioned values. In several embodiments, the buffered solution has a pH between 8.0 to 10.5 (or about 8.0 to about 10.5). In several embodiments, the pH of the buffered solution may be from 8.9 to 9.5 (or about 8.9 to about 9.5), from 9.0 to 9.4 (or about 9.0 to about 9.4), or from 9.1 to 9.3 (or about 9.1 to about 9.3). In several embodiments, the pH of the buffered solution may be 9.2 (or about 9.2).

In several embodiments, a crosslinking agent is provided in the solution at a concentration (in w/v) of equal to or less than about: 1%, 2.5%, 5.0%, 7.5%, 10%, 12.5%, or ranges including and/or spanning the aforementioned values. In several embodiments, the crosslinking agent is provided in the solution at a concentration ranging from 1% to 10% (or from about 1% to about 10%) (w/v), from 2% to 8% (or from about 2% to about 8%) (w/v), from 3% to 7% (or about 3% to about 7%) (w/v), or from 4% to 6% (or about 4% to about 6%) (w/v). In some embodiments, the crosslinking agent concentration in the solution is 4% (or about 4%) (w/v).

In several embodiments, the collagen-based material is exposed to the alkaline crosslinking conditions (e.g., is placed in the solution comprising crosslinking agent) for a period of time to provide a crosslinked collagen-based material. In several embodiments, the crosslinking reaction is allowed to proceed for equal to or at least about: 100 hours, 140 hours, 150 hours, 152 hours, 155 hours, 157.5 hours, 160 hours, 165 hours, or ranges including and/or spanning the aforementioned values.

In several embodiments, as shown in FIG. 3 step (1001), the collagen-based material is exposed to alkaline conditions or crosslinking to achieve full crosslinking of the material. In such an embodiment, the crosslinks are primarily amine-based (e.g., involve amine groups of the collagen material). In several embodiments, as an alternative to a crosslinking reaction that occurs under only alkaline conditions (as in FIG. 3), the collagen material may also be exposed to acidic pH to achieve partial crosslinking of the collagen-based material through carboxylic acids (as shown in Step (1003) of FIG. 4 and provided in Formula (Id)).

As shown in FIG. 4, where a mixture of pH conditions is used, a first and second crosslinking step can be used. Step 1002 involves exposing the collagen-based material to a first buffered solution comprising a first crosslinking agent at a first pH for a first period of time to provide a partially crosslinked collagen-based material. In some embodiments, the first pH is high enough to result in crosslinks that are primarily amine-based. In some embodiments, the first buffered solution has a pH between 8.0 to 10.5 (or about 8.0 to about 10.5). In some embodiments, the pH of the first buffered solution may be from 8.9 to 9.5 (or about 8.9 to about 9.5), from 9.0 to 9.4 (or about 9.0 to about 9.4), or from 9.1 to 9.3 (or about 9.1 to about 9.3). In some embodiments, the pH of the first buffered solution may be 9.2 (or about 9.2). The concentration of first crosslinking agent in the first solution may be from 1% to 10% (or from about 1% to about 10%) (w/v), from 2% to 8% (or from about 2% to about 8%) (w/v), from 3% to 7% (or about 3% to about 7%) (w/v), or from 4% to 6% (or about 4% to about 6%) (w/v). In some embodiments, the first crosslinking agent concentration in the first solution is 4% (or about 4%) (w/v). The first period of time for the crosslinking reaction depends on the desired level of crosslinking, and may be from 0.5 hours to 64 hours (or about 0.5 hours to about 64 hours). In some embodiments, the first period of time may be from 1 hour to 60 hours (or about 1 hour to about 60 hours), from 10 hours to 50 hours (or about 10 hours to about 50 hours), or from 20 hours to 40 hours (or about 20 hours to about 40 hours). In some embodiments the first period of time may be from 0.5 hours to 10 hours (or about 0.5 hours to about 10 hours), from 10 hours to 20 hours (or about 10 hours to about 20 hours), from 20 hours to 30 hours (or about 20 hours to about 30 hours), from 30 hours to 40 hours (or about 30 hours to about 40 hours), from 40 hours to 50 hours (or about 40 hours to about 50 hours), from 50 hours to 60 hours (or about 50 hours to about 60 hours), or from 60 hours to 64 hours (or about 60 hours to about 64 hours). In some embodiments, the partially crosslinked collagen-based material comprises partially crosslinked collagen strands, and the crosslinks are primarily amine-based crosslinks.

Step 1003 involves exposing the collagen-based material to a second buffered solution comprising a second crosslinking agent at a low pH for a second period of time to provide a tailorably crosslinked collagen-based material. The pH of the second buffered solution is low enough to result in crosslinks that are primarily ester-based. In some embodiments, the pH of the second buffered solution may be from 3.0 to 5.5 (or about 3.0 to about 5.5). In some embodiments, the pH of the second buffered solution may be from 4.2 to 4.8 (or about 4.2 to about 4.8), from 4.3 to 4.7 (or about 4.3 to about 4.7), or from 4.4 to 4.6 (or about 4.4 to about 4.6). In some embodiments, the pH of the second buffered solution may be 4.5 (or about 4.5). The concentration of second crosslinking agent in the second solution may be from 1% to 10% (or from about 1% to about 10%) (w/v), from 2% to 8% (or from about 2% to about 8%) (w/v), from 3% to 7% (or about 3% to about 7%) (w/v), or from 4% to 6% (or about 4% to about 6%) (w/v). In some embodiments, the first crosslinking agent concentration in the first solution is 4% (or about 4%) (w/v). The second period of time for the crosslinking may be from 100 hours to 160 hours (or about 100 hours to about 160 hours). In some embodiments, the second period of time for the crosslinking may be from 100 hours to 170 hours (or about 100 hours to about 170 hours), from 110 hours to 160 hours (or about 110 hours to about 160 hours), from 120 hours to 150 hours (or about 120 hours to about 150 hours), or from 130 hours to 140 hours (or about 130 hours to about 140 hours). In some embodiments, the second period of time for the crosslinking may be from 100 hours to 110 hours (or about 100 hours to about 110 hours), from 110 hours to 120 hours (or about 110 hours to about 120 hours), from 120 hours to 130 hours (or about 120 hours to about 130 hours), from 130 hours to 140 hours (or about 130 hours to about 140 hours), from 140 hours to 150 hours (or about 140 hours to about 150 hours), from 150 hours to 160 hours (or about 150 hours to about 160 hours), or from 160 to 170 hours (or about 160 hours to about 170 hours). In some embodiments, the second period of time for the crosslinking may be performed for a period that exceeds 170 hours.

In some embodiments, for a procedure using more than one pH condition as shown in FIG. 3, the total exposure time to the first and second buffered solutions (the total of the first period of time and the second period of time) is such that the resulting tailorably crosslinked collagen-based material is substantially fully crosslinked. In some embodiments, the total exposure time will be sufficient to afford a material that contains a small enough amount of pendant free epoxides such that the material is biocompatible. In some embodiments, the sum of the first period of time and the second period of time is from 100.5 hours to 110 hours (or about 100.5 hours to about 110 hours), from 110 hours to 120 hours (or about 110 hours to about 120 hours), from 120 hours to 130 hours (or about 120 hours to about 130 hours), from 130 hours to 140 hours (or about 130 hours to about 140 hours), from 140 hours to 150 hours (or about 140 hours to about 150 hours), and/or from 150 hours to 160 hours (or about 150 hours to about 160 hours). In some embodiments, the sum of the first period of time and the second period of time is 160 hours (or about 160 hours). In some embodiments, the sum of the first and second periods of time is longer than 160 hours.

Alternatively, the pH of the buffered solutions and reaction times in steps 1002 and 1003 may be reversed in some embodiments. In some embodiments, step 1002 is performed before step 1003. In other embodiments, not shown, step 1002 may follow step 1003. The collagen-base material may be exposed to a crosslinking agent solution with a low pH first, and then a second crosslinking agent solution with a high pH second. In this case, the first buffered solution has a low pH, while the second buffered solution has a high pH. In other embodiments, as shown in FIG. 3, the low pH condition step can be omitted. For example, the crosslinking can be performed under only alkaline conditions, as shown in FIG. 3.

In some embodiments, the crosslinking agent (e.g., in steps 1002 and/or 1003) may be a diepoxide. In some embodiments, the crosslinking agent (e.g., in steps 1002 and/or 1003) is selected from the group consisting of glycol diglycidyl ether, glycerol diglycidyl ether, glycerol triglycidyl ether, and butanediol diglycidyl ether. In some embodiments, where two crosslinking conditions are used (e.g., in steps 1002 and/or 1003), the crosslinking agents in steps 1002 and/or 1003 are the same. In other embodiments, the crosslinking agents (e.g., in steps 1002 and/or 1003) may be different. In several embodiments, a plurality of crosslinking agents can be used in each crosslinking step (e.g., crosslinking agents independent selected from the group consisting of glycol diglycidyl ether, glycerol diglycidyl ether, glycerol triglycidyl ether, and butanediol diglycidyl ether). In some embodiments, where two crosslinking conditions are used (e.g., in steps 1002 and/or 1003), the crosslinking agent in both conditions is BDDGE. In several embodiments, where one crosslinking condition is used, the crosslinking agent is BDDGE. In some embodiments, the crosslinking agents (e.g., in steps 1002 and/or 1003) are water soluble, non-polymeric epoxies such as polyol polyglycidylethers.

Several embodiments of achieve a predetermined degree of crosslinking by precise control of the concentration of the crosslinking agent, the pH of the crosslinking agent, the length of time the collagen-based material is exposed to the crosslinking agent, and the temperature at which the collagen-based material is exposed to the crosslinking agent. One degree of crosslinking would be crosslinking only about 50% of the free amine or carboxyl groups which would enable the collagen material to retain sufficient resistance to premature enzymatic degradation and retain sufficient strength to complete its intended therapeutic role, yet allow the covering to ultimately dissolve, thereby avoiding a permanent implant.

Step 1004 involves isolating the tailorably crosslinked collagen-based material to provide a crosslinked collagen-based material. In several embodiments, the crosslinked material is rinsed (as shown in Step 1004 of FIGS. 3 and 4). In several embodiments, the rinse is performed in water (e.g., deionized or distilled). In several embodiments, the crosslinked collagen-based material is then rinsed with or bathed in a quenching solution (Step 1005). In several embodiments, the solution comprises a quenching group. In several embodiments, the quenching group (or quenching agent) may be represented by H—X¹—R² (as disclosed elsewhere herein). In several embodiments, the quenching group (e.g., quenching agent) is an alcohol. In several embodiments, the quenching agent is ethanol (e.g., where X¹ is —O— and R² is —CH₂CH₃. In several embodiments, the quenching agent is provided in a solution. In several embodiments, the quenching agent is provided in a solution of water. In several embodiments, the quenching agent is provided at a concentration in the solution of (v/v or w/v) of equal to or greater than about: 5%, 10%, 12.5%, 15%, 20%, 25%, 30%, 35%, or ranges including and/or spanning the aforementioned values. In several embodiments, after quenching, a treated crosslinked collagen-based material is provided.

In several embodiments, the treated crosslinked collagen-based material is dried (Step 1006). In several embodiments, the treated crosslinked collagen-based material is freeze dried. treated crosslinked collagen-based material is performed on final rinsed collagen in a multistep process designed to allow efficient rehydration of the material. In several embodiments, lyophilization is performed by freezing the treated crosslinked collagen-based material. In several embodiments, the temperature is ramped to <−30° C. under high vacuum and held for >1 Hour. In several embodiments, the temperature is ramped to <−20° C. under high vacuum and held for >2 Hour. In several embodiments, the temperature is ramped to <−5° C. under high vacuum and held for >1 Hour. In several embodiments, the temperature is ramped to 20°−25° C. under high vacuum and hold for >1 Hour.

In several embodiments, the treated crosslinked collagen-based material can be rehydrated after drying (as shown in Step 1007). In several embodiments, surprisingly, the treated crosslinked collagen-based material retains its material properties after rehydration. In several embodiments, once rehydrated the treated crosslinked collagen-based material is flexible and conforms to the shape of anatomical feature after to perform well in the surgical application of the material. In several embodiments, the product (e.g., treated crosslinked collagen-based material) is provided in a sheet form or assembled using a reinforcing fabric. In several embodiments, the product is the packaged in a double pouched radiation proof and moisture barrier peel pouch. In several embodiments, the treated crosslinked collagen-based material is sterilized. In several embodiments, the treated crosslinked collagen-based material is sterilized using e-beam conditions (e.g., with a dose of 20-30 kGy).

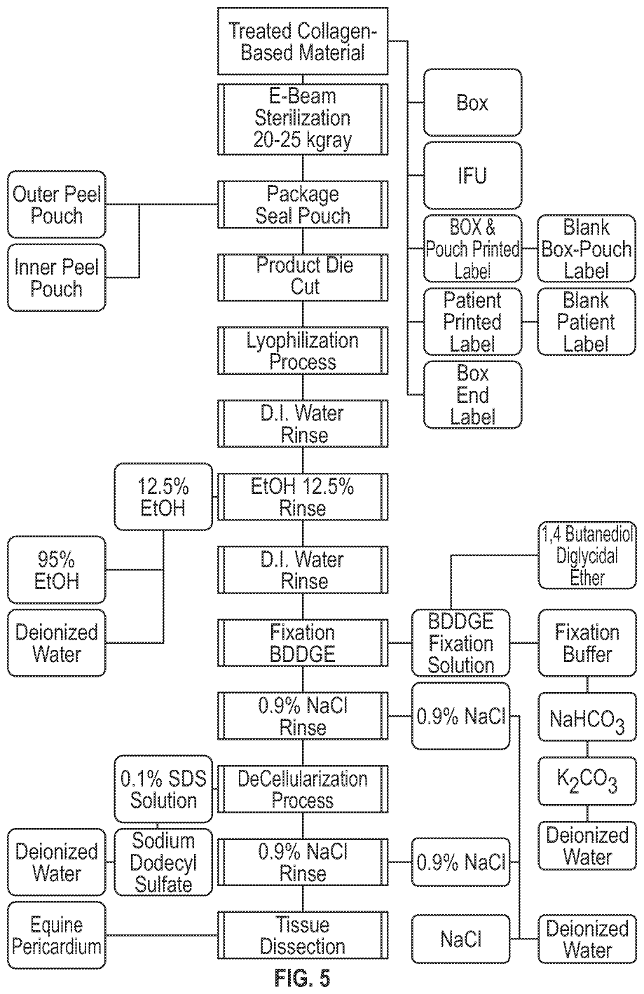

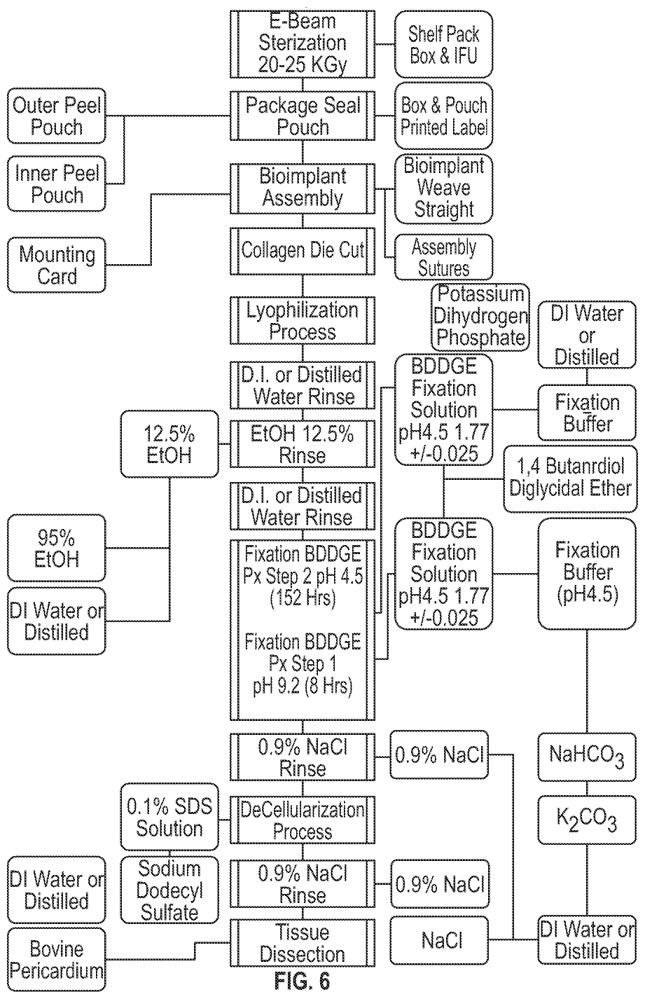

Alkaline crosslinking (e.g., of FIG. 3) provides primarily or substantially only amine based crosslinks with longer degradation times. Alkaline crosslinking may be intended for situations where long term intact collagen or implant of the device is preferable. Since the alkaline crosslinking process yields a material that is very resistant is biologic degradation it is suitable for surgical repair where natural biologic remodeling may be very slow or very long term. Examples include repair of diabetic extremity ulcers and soft tissue repair. Ester and amine crosslinked collagen matrices (as provided in FIG. 4) may be for situations where short term contact or implant of the device is preferable. Since the ester/amine process yields a material that is not as resistant to biologic degradation, it is suitable for surgical repair where biologic remodeling is considered beneficial early term. A detailed example of the manufacturing process for FIG. 3 is shown in FIG. 5. A detailed example of the manufacturing process for FIG. 4 is shown in FIG. 6.

### EXAMPLES

### Materials and Methods

The following is a list of materials and equipment used throughout the Examples section: Perkin Elmer Model DSC 4000, Differential Scanning calorimeter; Boekel Scientific Oven Model 132000; Mettler Toledo Analytic Balance Model AL54; Mettler Toledo Balance New Classic ML; Calipers, Mitutoyo Corp., 505-626 Dial Caliper; Labconco Lyophilizer Model Freezone 6 with Tray Dryer; Scalpel, Bard-parker Stainless Steel Sterile blade #10; Sklar Tru-Punch, Disposable Biopsy Punch; VWR SympHony SB70P pH Meter; Distilled Water; Sodium Dodecyl Sulfate Solution 0.1%; Sonic Bath=Bransonic 2510; Equine Pericardium; 0.9% NaCl Solution; 1,4 Butanediol Diglycidyl Ether; Standard HEPES Buffer; 0.1M Phosphate Buffer pH 4.5 (purchased from Teknova; Cat. #: P4000); Fixation Buffer pH 9.2.

### Preparation of Pericardium to Form Collagen-Based Material:

Equine pericardial sacks were procured fresh from Carnicos de Jerez S. A. de C. V. and air freighted in 0.9% NaCl solution on ice. Immediately on receipt, all sacks were rinsed in fresh, cold 0.9% NaCl solution, debrided of fat and excess fibrous tissue, and trimmed with a surgical scalpel to create 8 similar patches approximately 10 cm×15 cm. All patches were decellularized by a process of 20 minutes sonication in a 0.1% solution of Sodium Dodecyl Sulfate (SDS) followed by three separate rinses in 500 ml of 0.9% NaCl solution to remove excess SDS. The decellularization process is intended to remove any excess intracellular materials. The anionic surfactant (SDS) used in the process also helps to reduce excess fats and oils. The treatment of the resulting patches yielded debrided, decellularized pericardial patches. One of these patches was set aside as a control for crosslinking experiments. Though equine pericardial sacks were used, the pericardium of bovine specimens, humans, or other mammals may be used.

### Example 1: Crosslinked Collagen-Based Material Preparation Using Low pH

To a solution phosphate buffered solution (PBS, 0.5 L, 4.5±0.2 pH) was added 1,4 butanediol diglycidyl ether (20 g) to afford a 4% by weight solution of 1,4 butanediol diglycidyl ether. This solution was stirred to yield a homogeneous solution of 4.5±0.2 pH 1,4 butanediol diglycidyl ether. This preparation provided a low pH solution of 1,4 butanediol diglycidyl ether. To the low pH solution of 1,4 butanediol diglycidyl ether (4.0% w/v 1,4 butanediol diglycidyl ether, 4.5±0.2 pH, excess) was added, an approximately 10×15 cm debrided, decellularized pericardial patch. The patch was allowed to remain in the 1,4 butanediol diglycidyl ether/PBS solution for 150-160 hours at which time the patch was rinsed with distilled water thoroughly. The patch was then rinsed with an alcohol quench solution comprising 12.5% to 25% ethanol in water. After the quench was performed, the patch was freeze dried.

### Example 2: Crosslinked Collagen-Based Material Preparation Using High pH and Low pH

To 1 L of deionized water was added potassium carbonate (6.5 grams) and sodium bicarbonate (16.6 grams). The solution was mixed until all solids dissolved. The pH of the solution was measured using a pH meter wherein the target pH was 9.2±0.2. If necessary, the pH of the solution was adjusted to 9.2±0.2 by adding dilute NaOH or dilute HCl. Next, to the buffered solution (bicarbonate buffer) was added 1,4 butanediol diglycidyl ether (40 g) to afford a 4% by weight solution of 1,4 butanediol diglycidyl ether. This solution was stirred to give a homogenous solution of 9.2±0.2 pH 1,4 butanediol diglycidyl ether. This preparation provided a high pH solution of 1,4 butanediol diglycidyl ether. To the high pH solution of 1,4 butanediol diglycidyl ether (4.0% w/v 1,4 butanediol diglycidyl ether, 9.2±0.2 pH, excess) was added, an approximately 10×15 cm debrided, decellularized pericardial patch. The patch was allowed to remain in the 4.0% w/v 1,4 butanediol diglycidyl ether, 9.2±0.2 pH solution for 8 hours, at which time it was added to a low pH solution of 1,4 butanediol diglycidyl ether as prepared in Example 1 (4.0% w/v 1,4 butanediol diglycidyl ether, 4.5±0.2 pH, excess). After 150-160 total crosslinking reaction time hours, the patch was removed from the low pH 1,4 butanediol diglycidyl ether solution and was rinsed with distilled water thoroughly. The patch was then rinsed with an alcohol quench solution comprising 12.5% to 25% ethanol in water. After the quench was performed, the patch was freeze dried.

### Example 3: Crosslinked Collagen-Based Material Preparation Using High pH and Low pH

To a high pH solution of 1,4 butanediol diglycidyl ether as prepared in Example 2 (4.0% w/v 1,4 butanediol diglycidyl ether, 9.2±0.2 pH, excess) was added, an approximately 10×15 cm debrided, decellularized pericardial patch. The patch was allowed to remain in the 4.0% w/v 1,4 butanediol diglycidyl ether, 9.2±0.2 pH solution for 24 hours, at which time it was added to a low pH solution of 1,4 butanediol diglycidyl ether as prepared in Example 1 (4.0% w/v 1,4 butanediol diglycidyl ether, 4.5±0.2 pH, excess). After 150-160 total crosslinking reaction time hours, the patch was removed from the low pH 1,4 butanediol diglycidyl ether solution and was rinsed with distilled water thoroughly. The patch was then rinsed with an alcohol quench solution comprising 12.5% to 25% ethanol in water. After the quench was performed, the patch was freeze dried.

### Example 4: Crosslinked Collagen-Based Material Preparation Using High pH and Low pH

To a high pH solution of 1,4 butanediol diglycidyl ether as prepared in Example 2 (4.0% w/v 1,4 butanediol diglycidyl ether, 9.2±0.2 pH, excess) was added, an approximately 10×15 cm debrided, decellularized pericardial patch. The patch was allowed to remain in the 4.0% w/v 1,4 butanediol diglycidyl ether, 9.2±0.2 pH solution for 36 hours, at which time it was added to a low pH solution of 1,4 butanediol diglycidyl ether as prepared in Example 1 (4.0% w/v 1,4 butanediol diglycidyl ether, 4.5±0.2 pH, excess). After 150-160 total crosslinking reaction time hours, the patch was removed from the low pH 1,4 butanediol diglycidyl ether solution and was rinsed with distilled water thoroughly. The patch was then rinsed with an alcohol quench solution comprising 12.5% to 25% ethanol in water. After the quench was performed, the patch was freeze dried.

### Example 5: Crosslinked Collagen-Based Material Preparation Using High pH and Low pH

To a high pH solution of 1,4 butanediol diglycidyl ether as prepared in Example 2 (4.0% w/v 1,4 butanediol diglycidyl ether, 9.2±0.2 pH, excess) was added, an approximately 10×15 cm debrided, decellularized pericardial patch. The patch was allowed to remain in the 4.0% w/v 1,4 butanediol diglycidyl ether, 9.2±0.2 pH solution for 48 hours, at which time it was added to a low pH solution of 1,4 butanediol diglycidyl ether as prepared in Example 1 (4.0% w/v 1,4 butanediol diglycidyl ether, 4.5±0.2 pH, excess). After 150-160 total crosslinking reaction time hours, the patch was removed from the low pH 1,4 butanediol diglycidyl ether solution and was rinsed with distilled water thoroughly. The patch was then rinsed with an alcohol quench solution comprising 12.5% to 25% ethanol in water. After the quench was performed, the patch was freeze dried.

### Example 6: Crosslinked Collagen-Based Material Preparation Using High pH and Low pH

To a high pH solution of 1,4 butanediol diglycidyl ether as prepared in Example 2 (4.0% w/v 1,4 butanediol diglycidyl ether, 9.2±0.2 pH, excess) was added, an approximately 10×15 cm debrided, decellularized pericardial patch. The patch was allowed to remain in the 4.0% w/v 1,4 butanediol diglycidyl ether, 9.2±0.2 pH solution for 64 hours, at which time it was added to a low pH solution of 1,4 butanediol diglycidyl ether as prepared in Example 1 (4.0% w/v 1,4 butanediol diglycidyl ether, 4.5±0.2 pH, excess). After 150-160 total crosslinking reaction time hours, the patch was removed from the low pH 1,4 butanediol diglycidyl ether solution and was rinsed with distilled water thoroughly. The patch was then rinsed with an alcohol quench solution comprising 12.5% to 25% ethanol in water. After the quench was performed, the patch was freeze dried.

### Example 7: Crosslinked Collagen-Based Material Preparation Using High pH

To a high pH solution of 1,4 butanediol diglycidyl ether as prepared in Example 2 (4.0% w/v 1,4 butanediol diglycidyl ether, 9.2±0.2 pH, excess) was added, an approximately 10×15 cm debrided, decellularized pericardial patch. The patch was allowed to remain in the 4.0% w/v 1,4 butanediol diglycidyl ether, 9.2±0.2 pH solution for 150-160 hours, at which time the patch was removed from the low pH 1,4 butanediol diglycidyl ether solution and was rinsed with was rinsed with distilled water thoroughly. The patch was then rinsed with an alcohol quench solution comprising 12.5% to 25% ethanol in water.

### Example 8: Drying Process

The patches prepared in Examples 1-7 were freeze-dried by lyophilization. The patches were subject to a multistage drying process. For the process, the patch was frozen in a lyophilization chamber. In the first lyophilization step the temperature was ramped to <−30° C. under high vacuum and held for >1 Hour. In the second lyophilization step the temperature was ramped to <−20° C. under high vacuum and held for >2 Hour. In the third lyophilization step the temperature was ramped to <−5° C. under high vacuum and held for >1 Hour. In the fourth lyophilization step the temperature was ramped to 20°−25° C. under high vacuum and hold for >1 Hour. The chamber and patch were then ramped to ambient temperature and pressure. Mechanical testing of a representative sample showed a tensile strength of greater than 10N with a shrinkage temperature of >76° C. Surprisingly, these dehydration conditions preserved the properties of the crosslinked collagen material (including tensile strength, shrinkage temperature, etc.). Also rehydration time in the operating room using sterile saline, flexibility & suture ability. Moisture content <12% to prevent microbial growth in storage

TABLE 4
Processing of Collagen Based Material.

| Materials: | BDDGE Fixed Equine Pericardium (P/N 30-0001) |
|---|---|
| Processing: | Lyophilization Cycle Parameters |
| | Pre freeze BDDGE Fixed Pericardial Sheets to ≤−5° C. |
| | Phase 1 - Ramp to <−30° C. under |
| | high vacuum and hold for >1 Hour |
| | Phase 2 - Ramp to <−20° C. under |
| | high vacuum and hold for >2 Hour |
| | Phase 3 - Ramp to <−5° C. under |
| | high vacuum and hold for >1 Hour |
| | Phase 4 - Ramp to 20°-25° C. under |
| | high vacuum and hold for >1 Hour |
| | Phase 5 - Ramp to ambient temperature and pressure |
| Size: | Per Tissue Mounting Frame TL-0005 |
| | (Approx. 20 cm × 12 cm) |
| Appearance: | Dry and white to light yellow sheet |
| Characteristics: | Temperature of Shrinkage (DSC) Test >76° C. |
| | Tensile Strength Test >10N |

### Example 9: Testing of the Crosslinked Collagen-Based Materials

### Temperature of Shrinkage (Ts):

Three 3 mm diameter samples were cut from each of the resulting crosslinked materials from examples 1-7 and the control using a Skylar 3 mm biopsy punch. Each sample was sealed in a Perkin Elmer DSC volatile sample pan (0219-0062). An empty pan is run in parallel with the test sample in the Differential Scanning calorimeter (DSC). Through comparison of heat flow of the empty pan and test pan, the peak temperature of enthalpy indicates the transition temperature or temperature of shrinkage (Ts) of the sample expressed in ° C. Ts of the samples are compared to that of the control or non-cross-linked to determine the comparative level of amine cross-linking present. Table 1 contains the results of each sample, separated by each example number.

TABLE 1
Temperature of Shrinkage Results (° C.)

| Sample # | 1 | 2 | 3 | Ave | SD |
|---|---|---|---|---|---|
| Example 1 | 69.63 | 70.90 | 69.71 | 70.08 | 0.58 |
| Example 2 | 72.81 | 72.40 | 72.41 | 72.54 | 0.19 |
| Example 3 | 73.91 | 74.09 | 74.19 | 74.06 | 0.12 |
| Example 4 | 76.17 | 75.98 | 76.03 | 76.06 | 0.08 |
| Example 5 | 78.31 | 78.54 | 78.53 | 78.46 | 0.11 |
| Example 6 | 77.58 | 77.11 | 76.98 | 77.22 | 0.26 |
| Example 7 | 77.26 | 77.30 | 78.21 | 77.59 | 0.44 |
| Control | 69.19 | 68.94 | 68.55 | 68.89 | 0.26 |

### Pronase Digestion Assay:

Three 1 cm×1 cm samples were cut from each of examples 1-7 and the control and tested per MF3-00X Pronase Digestion. Per procedure MF3-00X each sample was placed in a 5 ml glass scintillation vial with 4 mls of a HEPES buffered solution with 95 mg/100 ml bacterial protease derived from Streptomyces griseus. The samples were incubated at 45° C. for 24 hours, blotted dry and lyophilized in the Labconco lyophilizer. Then each sample was weighed utilizing the Mettler Toledo analytic balance. All samples were reweighed using the Mettler Toledo analytic balance. The percent degradation was determined calculating the percent change in weight before and after 24 hours exposure to the protease. Table 2 contains the results of each sample, separated by each example number.

TABLE 2
Protease Digestion % After 24 Hours Exposure to Protease

| Sample # | | | | | |
|---|---|---|---|---|---|
| Wt. (mg) | | | | | |
| after/ | | | | Ave % | |
| before | 1 | 2 | 3 | Remaining | SD |
| Example 1 | 5.30/7.60 | 5.40/7.30 | 5.60/7.30 | 73.47% | 2.87 |
| Example 2 | 7.80/10.10 | 8.30/10.50 | 10.60/13.20 | 78.86% | 1.26 |
| Example 3 | 10.60/11.90 | 10.80/12.20 | 12.40/13.80 | 89.15% | 0.55 |
| Example 4 | 10.50/11.90 | 11.30/12.90 | 12.70/13.90 | 89.07% | 1.65 |
| Example 5 | 14.30/14.80 | 15.70/16.00 | 19.80/20.50 | 97.11% | 0.72 |
| Example 6 | 5.00/5.40 | 6.10/6.40 | 5.10/5.50 | 93.54% | 1.25 |
| Example 7 | 11.50/12.00 | 12.10/12.60 | 12.60/12.90 | 96.51% | 0.83 |
| Control | 8.60/13.40 | 8.80/13.40 | 9.40/13.50 | 66.49% | 2.30 |

The degradation rate was then calculated by dividing the % digestion after 24 hours by 24 hours to yield % degraded/hour. Table 3 shows those results.

TABLE 3
Degradation Rate in %/hr.

| Sample # | 1 | 2 | 3 | Ave | SD |
|---|---|---|---|---|---|
| Example 1 | 1.26 | 1.08 | 0.97 | 1.11 | 0.15 |
| Example 2 | 0.95 | 0.87 | 0.82 | 0.88 | 0.06 |
| Example 3 | 0.46 | 0.48 | 0.42 | 0.45 | 0.03 |
| Example 4 | 0.49 | 0.52 | 0.36 | 0.46 | 0.08 |
| Example 5 | 0.14 | 0.08 | 0.14 | 0.12 | 0.04 |
| Example 6 | 0.31 | 0.20 | 0.30 | 0.27 | 0.06 |
| Example 7 | 0.17 | 0.17 | 0.10 | 0.15 | 0.04 |
| Control | 1.49 | 1.43 | 1.27 | 1.40 | 0.12 |

The study demonstrated that a pH shift from high (9.2) to low (4.5) within the first 64 hours of a 160 hour 4% 1,4 butanediol diglycidyl ether cross-linking process resulted in an extracellular collagen matrix with progressively lower Ts values, lower resistance to protease and a significantly faster bioresorbtion rate.

The process of pH modulation of the 1,4 butanediol diglycidyl ether cross-linking process of extracellular collagen matrix material is a feasible method of producing a medical device for general surgical repair with a controlled predetermined bioresorbtion rate.

### Measuring Free Amine Content

In addition to the tests described below, amine content can also be calculated. In some embodiments, the free amine group content of tailorably crosslinked collagen-based material, expressed as a percentage of the collagen-based material (%), can be determined using a 2,4,6-trinitrobenzenesulfonic acid (TNBS; 1.0 M solution in water, Fluka, Buchs, Switzerland) colorimetric assay. To a sample of 2-4 milligrams (mg) of tailorably crosslinked collagen-based material a solution of 1 ml of a 4% (weight/volume) aqueous NaHCO₃ (pH 9.0; Aldrich, Bornem, Belgium) solution and 1 ml of a freshly prepared 0.5% (weight/volume) aqueous TNBS solution can be added. After reaction for 2 hours at 40° C., 3.0 ml of 6 M HCl (Merck, Darmstadt, Germany) can be added and the temperature can be raised to 60° C. When complete solubilization of tailorably crosslinked collagen-based material is achieved, the resulting solution is diluted with 15 ml of deionized water and the absorbance was measured on a Hewlett-Packard HP8452A UV/VIS spectrophotometer at a wavelength of 345 nm. A control is prepared applying the same procedure except that HCl was added before the addition of TNBS. The free amine group content is calculated using a molar absorption coefficient of 14600 1 mol⁻¹ cm⁻¹ for trinitrophenyl lysine [Wang C. L., et al., Biochim. Biophys. Acta, 544, 555-567, (1978)].

The free amine group content of tailorably crosslinked collagen-based material also can be determined using a ninhydrin test. The following describes the general procedures for testing the amine content of a collagen-based material. Briefly, a sample of 1-25 milligrams (mg) of tailorably crosslinked collagen-based material is collected. Next, a solution of 1 ml of a 4% (weight/volume) ninhydrin in methyl cellosolve is prepared. Then a 0.2 M sodium citrate buffer is prepared by dissolving 1.05 g of citric acid monohydrate and 0.04 g of stannous chloride dihydrate in 11 mL of 1.0 N NaOH and adding 14 mL of purified water. The pH of the sodium citrate buffer is adjusted to 4.9 to 5.1 with HCl and/or NaOH. Next, the 4% ninhydrin solution and sodium citrate buffer are mixed in a dark bottle for immediate use. Now a solution of N-acetyllysine (ALys) is prepared by dissolving 47.1 mg of ALys in 50 mL of purified water. The ALys is used as a standard solution for calibrating the absorbance which is read at 570 nm. After a standard curve is plotted, samples of dried tissue are tested. Each solution for to be read by absorbance is prepared using 1 mL of buffered ninhydrin, 100 microliters of purified water, and the tissue or control sample. The test solutions are heated to 100° C. for 20 minutes, cooled, then 5 mL of isopropyl alcohol is added. The absorbance is then read and the amount of mols of amine per gram of sample and control is calculated from using the following equation: A=mX+b where, A=absorbance, X=content of ALys in micromoles, m=the slope, and b=the y-intercept. The content of micromoles of free amine in the sample is then Xₛₐₘₚ=(Aₛₐₘₚ−b)/M.

### Mechanical Properties:

Stress-strain curves of the degradable bioprosthesis can be taken using uniaxial measurements using a mechanical tester. Tensile bars (40.0 mm×4.0 mm×1.4 mm) can be cut using a dumb-bell shaped knife and can be hydrated for at least one hour in PBS at room temperature. The thickness of the samples can be measured in triplicate using a spring-loaded type micrometer (Mitutoyo, Tokyo, Japan). An initial gauge length of 10 mm was used and a crosshead speed of 5 mm/minute can be applied until rupture of the test specimen occurs. A preload of 0.05 N can be applied to prestretch the specimen before the real measurement. The tensile strength, the elongation at alignment, the elongation at break, the low strain modulus and the high strain modulus of the sample can be calculated from five independent measurements.

Various modifications to the implementations described in this disclosure may be readily apparent to those skilled in the art, and the generic principles defined herein may be applied to other implementations without departing from the spirit or scope of this disclosure. Thus, the disclosure is not intended to be limited to the implementations shown herein, but is to be accorded the widest scope consistent with the principles and features disclosed herein. Certain embodiments of the invention are encompassed in the claim set listed below.

While certain embodiments of the inventions have been described, these embodiments have been presented by way of example only, and are not intended to limit the scope of the disclosure. Indeed, the novel methods and systems described herein may be embodied in a variety of other forms. Furthermore, various omissions, substitutions and changes in the systems and methods described herein may be made without departing from the spirit of the disclosure. The accompanying claims and their equivalents are intended to cover such forms or modifications as would fall within the scope and spirit of the disclosure. Accordingly, the scope of the present inventions is defined only by reference to the appended claims.

Features, materials, characteristics, or groups described in conjunction with a particular aspect, embodiment, or example are to be understood to be applicable to any other aspect, embodiment or example described in this section or elsewhere in this specification unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The protection is not restricted to the details of any foregoing embodiments. The protection extends to any novel one, or any novel combination, of the features disclosed in this specification (including any accompanying claims, abstract and drawings), or to any novel one, or any novel combination, of the steps of any method or process so disclosed.

Furthermore, certain features that are described in this disclosure in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations, one or more features from a claimed combination can, in some cases, be excised from the combination, and the combination may be claimed as a subcombination or variation of a subcombination.

Moreover, while operations may be depicted in the drawings or described in the specification in a particular order, such operations need not be performed in the particular order shown or in sequential order, or that all operations be performed, to achieve desirable results. Other operations that are not depicted or described can be incorporated in the example methods and processes. For example, one or more additional operations can be performed before, after, simultaneously, or between any of the described operations. Further, the operations may be rearranged or reordered in other implementations. Those skilled in the art will appreciate that in some embodiments, the actual steps taken in the processes illustrated and/or disclosed may differ from those shown in the figures. Depending on the embodiment, certain of the steps described above may be removed, others may be added. Furthermore, the features and attributes of the specific embodiments disclosed above may be combined in different ways to form additional embodiments, all of which fall within the scope of the present disclosure. Also, the separation of various system components in the implementations described above should not be understood as requiring such separation in all implementations, and it should be understood that the described components and systems can generally be integrated together in a single product or packaged into multiple products.

For purposes of this disclosure, certain aspects, advantages, and novel features are described herein. Not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Conditional language, such as “can,” “could,” “might,” or “may,” unless specifically stated otherwise, or otherwise understood within the context as used, is generally intended to convey that certain embodiments include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting, whether these features, elements, and/or steps are included or are to be performed in any particular embodiment.

Conjunctive language such as the phrase “at least one of X, Y, and Z,” unless specifically stated otherwise, is otherwise understood with the context as used in general to convey that an item, term, etc. may be either X, Y, or Z. Thus, such conjunctive language is not generally intended to imply that certain embodiments require the presence of at least one of X, at least one of Y, and at least one of Z.

Language of degree used herein, such as the terms “approximately,” “about,” “generally,” and “substantially” as used herein represent a value, amount, or characteristic close to the stated value, amount, or characteristic that still performs a desired function or achieves a desired result. For example, the terms “approximately”, “about”, “generally,” and “substantially” may refer to an amount that is within less than 10% of, within less than 5% of, within less than 1% of, within less than 0.1% of, and within less than 0.01% of the stated amount. As another example, in certain embodiments, the terms “generally parallel” and “substantially parallel” refer to a value, amount, or characteristic that departs from exactly parallel by less than or equal to 15 degrees, 10 degrees, 5 degrees, 3 degrees, 1 degree, or 0.1 degree.

What is claimed is:

1. An orthopedic implant for treatment of a distal extremity joint, the orthopedic implant comprising:
a support element comprising:
a first end portion;
a second portion; and
a middle portion between the first end portion and the second end portion, the middle portion having a first main surface having a first width, a second main surface opposite to the first main surface, a first side edge surface, and a second side edge surface; and
a crosslinked collagen-based covering positioned around at least part of a length of the middle portion of the support element so that the crosslinked collagen-based covering covers at least part of the length of the first main surface, the second main surface, and the first side edge of the middle portion of the support element, the crosslinked collagen-based covering comprising:
a collagenous substrate comprising collagen strands, a crosslink, and a quenched crosslinking agent;
wherein the crosslink comprises a crosslinking unit, a first amine, and a second amine, the first amine being part of a first collagen strand of the collagenous substrate and the second amine being part of a second collagen strand of the collagenous substrate, the crosslink being represented by Formula (I): 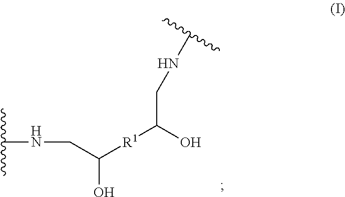
wherein the quenched crosslinking agent is bonded to the collagenous material through a third amine of the collagenous substrate and is represented by Formula (II): 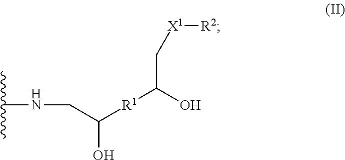
where
R¹ is selected from the group consisting of optionally substituted alkylene, optionally substituted polyether, and optionally substituted polyamino;
R² is selected from the group consisting of optionally substituted alkylene, optionally substituted polyether, and optionally substituted polyamino;
X¹ is selected from the group consisting of —O— and —NH— where each instance of “ [symbol] ” of Formulae (I) and (II) represents a portion of the collagenous substrate.

2. The orthopedic implant of claim 1, wherein the support element comprises at least one of a thread, a suture, a sheet, a strip, a fabric, and a weave.

3. The orthopedic implant of claim 1, wherein the support element comprises a braided material.

4. The orthopedic implant of claim 1, wherein the support element comprises a mesh material.

5. The orthopedic implant of claim 1, wherein the support element comprises a polymeric surgical mesh material.

6. An orthopedic implant for treatment of a soft tissue defect, the orthopedic implant comprising:
a braided support element comprising:
a first end portion;
a second portion; and
a middle portion between the first end portion and the second end portion, the middle portion having a first main surface having a first width, a second main surface opposite to the first main surface, a first side edge surface, and a second side edge surface; and
a crosslinked collagen-based covering positioned around at least part of a length of the middle portion of the support element so that the crosslinked collagen-based covering covers at least part of the length of the first main surface, the second main surface, and the first side edge of the middle portion of the support element, the crosslinked collagen-based covering comprising:
a collagenous substrate comprising collagen strands, a crosslink, and a quenched crosslinking agent;
wherein the crosslink comprises a crosslinking unit, a first amine, and a second amine, the first amine being part of a first collagen strand of the collagenous substrate and the second amine being part of a second collagen strand of the collagenous substrate, the crosslink being represented by Formula (I): 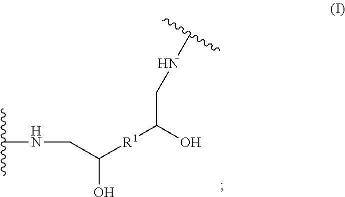
wherein the quenched crosslinking agent is bonded to the collagenous material through a third amine of the collagenous substrate and is represented by Formula (II): 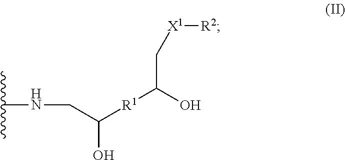
where
R¹ is selected from the group consisting of optionally substituted alkylene, optionally substituted polyether, and optionally substituted polyamino;
R² is selected from the group consisting of optionally substituted alkylene, optionally substituted polyether, and optionally substituted polyamino;
X¹ is selected from the group consisting of —O— and —NH—
where each instance of “ [symbol] ” of Formulae (I) and (II) represents a portion of the collagenous substrate.

7. The orthopedic implant of claim 6, wherein the orthopedic implant is sized and configured for use in an abdominal hernia treatment procedure.

8. The orthopedic implant of claim 6, wherein the orthopedic implant is configured for tendon, ligament, and/or other tissue repair, support, reconstruction and/or other treatment of a patient's hip or spine.

9. The orthopedic implant of claim 6, wherein the orthopedic implant is configured for tendon, ligament, and/or other tissue repair, support, reconstruction and/or other treatment of any of a patient's spine.

10. The orthopedic implant of claim 6, wherein the crosslinked collagen-based covering does not cover the first end portion or the second end portion of the support element.

11. The orthopedic implant of claim 6, wherein the crosslinked collagen-based covering covers at least the first main surface, the second main surface, and the first side edge

12. The orthopedic implant of claim 6, wherein the crosslinked collagen-based covering is wrapped around at least the middle portion of the support element.

13. The orthopedic implant of claim 6, wherein the crosslinked collagen-based covering comprises a sheet of the collagenous substrate that is wrapped around at least the first main surface, the second main surface, and the first side edge surface of the middle portion of the support element.

14. The orthopedic implant of claim 6, wherein the crosslinked collagen-based covering comprises a sheet of the collagenous substrate that is wrapped continuously around the first main surface, the second main surface, the first side edge surface, and the second side edge surface of the middle portion of the support element.

15. The orthopedic implant of claim 6, wherein the crosslinked collagen-based covering comprises a sheet of the collagenous substrate that has at least one fold along a length thereof, the fold being configured to facilitate a more uniform height or thickness of the orthopedic implant along the middle portion of the support element.

16. The orthopedic implant of claim 15, wherein the crosslinked collagen-based covering comprises at least one cut along the at least one fold.

17. The orthopedic implant of claim 15, wherein the at least one fold is aligned with the first side edge surface.

18. The orthopedic implant of claim 15, wherein the crosslinked collagen-based covering comprises at least one channel having a reduced thickness along the at least one fold, the reduced thickness being less than a thickness of the crosslinked collagen-based covering adjacent to the at least one channel.

19. The orthopedic implant of claim 18, wherein the reduced thickness of the channel is from 0.05 mm to 0.15 mm and the thickness of the crosslinked collagen-based covering adjacent to the at least one channel is from 0.3 mm to 0.7 mm.

20. A method of making an orthopedic implant, comprising:
providing a collagen-based material;
exposing the collagen-based material to crosslinking conditions to provide a crosslinked collagen-based material;
exposing the crosslinked collagen-based material to a quenching agent to provide a treated crosslinked collagen-based material; and
coupling the treated crosslinked collagen-based material with at least a portion of a braided support element.
